(19) United States
(12) Patent Application Publication
(10) Pub. No.: US 2026/0168037 A1
(43) Pub. Date: Jun. 18, 2026

(54) CANCER SCREENING ENHANCEMENT

(71) Applicant: James J. Myrick, Saint Augustine, FL (US)

(72) Inventor: James J. Myrick, Saint Augustine, FL (US)

(21) Appl. No.: 19/535,784

(22) Filed: Feb. 10, 2026

Related U.S. Application Data
(63) Continuation of application No. 17/807,427, filed on Jun. 17, 2022, now abandoned.
Application No. 17/807,427 is a continuation-in-part of application No. 17/089,520, filed on Nov. 4, 2020, now abandoned.
Application No. 17/089,520 is a continuation of application No. 16/870,944, filed on May 9, 2020, now abandoned.
(60) Provisional application No. 62/845,665, filed on May 9, 2019.
(60) Provisional application No. 62/849,960, filed on May 20, 2019.
(60) Provisional application No. 63/211,773, filed on Jun. 17, 2021.

(51) Int. Cl. C12Q 1/6886; A61K 31/4706; A61K 31/7048; G01N 33/575

(52) U.S. Cl. CPC C12Q 1/6886; A61K 31/4706; A61K 31/7048; G01N 33/57585; C12Q 2600/156

(57) ABSTRACT

Provided herein is a method for detecting cancer in an asymptotic human patient not previously diagnosed with cancer. The method may comprise one or more of the following steps: (1) administering a short-term effective dose to the patient of a drug means for selectively inducing apoptosis and/or ferroptosis in human cancer cells; (2) discontinuing the administration of drug means for selectively inducing apoptosis and/or necroptosis in human cancer cells within 3 days of the initial administration; (3) obtaining a sample of a body fluid from the patient within 7 days of the initial administration of the apoptosis inducing drug means; and (4) analyzing the sample fluid from the patient for the presence of circulating tumor DNA and/or cancer-characteristic circulating biomarkers.

### CROSS REFERENCE

This application claims the benefit of the filing date of U.S. application Ser. No. 17/807,427 filed on Jun. 17, 2022, which claims the benefit of U.S. Provisional Application No. 63/211,773 filed on Jun. 17, 2021, which is a continuation in part of U.S. application Ser. No. 17/089,520 filed on Nov. 4, 2020, which is a continuation of Ser. No. 16/870,944 filed on May 9, 2020, which claims the benefit of U.S. Provisional Application Nos. 62/849,960 filed on May 20, 2019 and 62/845,665 filed on May 9, 2019, each of which is hereby incorporated by reference as if fully set forth herein.

### FIELD OF THE INVENTION

The present disclosure is directed to cancer screening, and more specifically to liquid biopsy methods for detecting early-stage human cancer, especially for asymptomatic patients who have not previously been diagnosed with cancer.

### BACKGROUND

To promote progress of science and the useful arts, to encourage research, and to reduce early cancer detection screening cost to save lives, a free license is hereby granted upon patent issuance for Medicare, Medicaid, Veterans Administration, and uninsured patients. A free license is hereby awarded upon patent grant for fully published medical research carried out at no charge to patients. A license fee of fifty cents per patient screening test ($0.50) is hereby granted for all other use.

Every year, cancer kills approximately 10 million people worldwide. Of those who die, two thirds do so because they were diagnosed with advanced disease. There is nearly universal agreement that the best way to abolish cancer's terrible impact on the world is through early detection.

Screening tests for people who have never been diagnosed as having cancer could provide early diagnosis with improved outcomes. Early detection of cancer before it has metastasized, significantly enlarged, or spread from its original location, can facilitate subsequent treatment to both remove or destroy the cancer, and limit or prevent metastasis. Beginning after the 1948 recognition of cell-free nucleic acids (cfNAs) in body fluids, methods for detection of cfNAs such as circulating tumor DNA (ctDNA), mRNA, and miRNAs from liquid biopsies have been sought and improved for cancer detection and diagnosis. Various conventional screening methods utilize detection of circulating tumor DNA (“ctDNA” herein), sloughed off from a small proportion of a patient's cancer cells (if present) which “shed” genetic material into the bloodstream, such as when they die. Such ctDNA can be analyzed and detected, even when mixed with the larger amounts of DNA fragments coming from the death of normal cells of the screening patient. Some ctDNA tests detect a wide range of DNA components, while other screening tests can utilize generic ctDNA properties such as methyl distributions and hydrophilic/hydrophobic properties of ctDNA as distinguished from normal cell DNA.

Significant medical and scientific effort has been devoted to cancer detection and epigenetic study. There are a number of liquid biopsy test methods which have been successfully developed and are known for detecting cancer in bodily fluids of human patients. Epigenomics AG, a molecular diagnostics company headquartered in Berlin, Germany with a wholly owned subsidiary, Epigenomics Inc. based in Seattle, WA, began offering a blood test in 2016 for colon cancer based on detecting genetic biochemical modification in cancer. Grail, Inc. is a Silicon Valley, CA company whose “mission is to detect cancer early, when it can be cured”. Grail has published and is further developing liquid biopsy (e.g., blood) tests to detect multiple types of cancer. Inivata, a Granta Park, Cambridge, UK-based liquid biopsy company, provides ctDNA testing from blood via its InVision™ liquid biopsy platform. Guardant Health, a Redwood City, CA biotechnology company also sells blood tests to track and, potentially detect, cancer including lung, breast, colorectal and ovarian cancer.

There is also significant, well-published conventional academic liquid biopsy cancer screening technology, such as the Johns Hopkins University CancerSEEK blood test designed to test for eight cancers. Moreover, University of Queensland researchers have developed a “10-minute test” called “methylscape” for early cancer detection based on detection of circulating cancer DNA in the bloodstream. An inexpensive test which determines ctDNA from a broad range of different types of cancer, such as the “methylscape” test is useful for affordability of broad general early cancer screening of the asymptomatic population.

Conventional assays and protocols provide sensitivity and specificity in the detection of ctDNA and its variations, but new sampling technology would be desirable.

While such conventional liquid biopsy screening tests have shown the ability to detect ctDNA and other malignancy markers, they have limited sensitivity for detecting very early stage cancer. It is important to have high sensitivity together with a low false positive rate for detecting cancer at a very early stage. Conventional ctDNA blood screening tests may require the presence of a relatively large tumor mass to shed enough ctDNA in the patient's blood for reliable early detection. A significant limitation for early cancer detection by liquid biopsy screening tests is the low ctDNA levels which are sloughed off, and introduced into a screening patient's bloodstream or other body fluids, from small early-stage cancers. Some calculations indicate that cancer tumors may need to be relatively large in order to shed enough ctDNA to be reliably detected by conventional ctDNA testing employing a standard 10 ml blood sample. There has been some concern that it may not be possible to detect more than about 65% to 70% of early cancers because many small tumors don't shed enough ctDNA into the blood. Even more problematic for early cancer detection, much of a screening patient's potential ctDNA, if present in apoptosing tumor cells, is lost before it can be sampled by liquid biopsy. Natural phagocytic processes engulf apoptotic cells to digest their contents for reuse and inflammation prevention. Moreover, circulating DNA which was not digested by phagocytotic processes, especially unprotected DNA, can be rapidly cleared from blood circulation. Accordingly, there is a need for new, improved methods for early-stage ctDNA cancer screening for asymptotic patients who have not been previously diagnosed with cancer.

### DESCRIPTION OF THE DRAWINGS

FIG. 1 is a schematic graph of circulating tumor DNA (ctDNA) concentration (and corresponding schematic human cancer tumor size/mass) in the blood of a generic cancer screening patient, versus time on a long timeline, as compared to a ctDNA detection limit in the patient's blood for conventional ctDNA test capabilities.

FIG. 2 is a schematic graph of circulating tumor DNA (ctDNA) concentration in the blood of a human cancer screening patient having a malignant tumor which is too small to produce adequate ctDNA concentration circulating in the patient's blood to be reliably detected by a conventional ctDNA liquid biopsy test. Upon single-dose or short-term administration of a cancer cell apoptosis-inducer, the ctDNA concentration can briefly surges to a level above its previous concentration prior to apoptosis-induction, which can be detected by ctDNA liquid biopsy test within the time period of ctDNA concentration surge, after which the ctDNA concentration may recede to pre-administration levels below the ctDNA test sensitivity in the absence of further drug administration.

FIG. 3 is a schematic block flow diagram of an embodiment of ctDNA and ctHistone sensitivity enhancement methods for liquid biopsy screening for asymptomatic patients.

### SUMMARY

There are many types of human cancer. Cancer treatments tend to focus on susceptibility of specific characteristics of a particular cancer type, so tend to be specific for different types of cancer. A broad screening test for asymptomatic patients should not be highly selectively limited in the types of cancer the test can detect. In accordance with the present disclosure, cancer screening methods are provided which can temporarily enhance the ctDNA concentration (and/or degradation cfNA products thereof, cancer marker RNA, and cancer marker proteins) produced in a screening patient's blood or other body fluid from an early stage or small tumor.

During apoptosis, chromatin compacts against the nuclear envelope for nuclear fragmentation into membrane-bound apoptotic bodies with minimal cell rupture, activation of caspases-3 and -7 which proceed to efferocytotic removal by phagocytic cells with little release into the bloodstream as compared to substantial release which occurs from cell death by necrosis, necroptosis, or pyroptosis. Accordingly much of the tumor dna of dying cancer cells is not released into the bloodstream, but is instead engulfed and digested by phagocytes such as macrocytes.

Accordingly, by briefly reducing the phagocytotic digestion of apoptotic cells, while briefly enhancing or otherwise enabling dysfunctional apoptosis of tumor cells, the amount and concentration of ctDNA in the screening patient's bloodstream as ctDNA can be briefly synergistically increased. Because of the complexity of the phagocytotic process, there are a variety of ways to reduce phagocytosis of apoptosing cancer cells. Phagocytotic digestion of apoptotic cells and cell bodies is triggered by the presence of phosphatidylserine in the outer leaflet of the plasma membrane of an apoptotic cell, which is externalized by scramplase proteins. TAM family kinase inhibitors administered to the patient can reduce scramblase externalization of phosphatidylserine, to reduce engulfment of apoptotic cells and cell bodies. Briefly administered inhibitors of TIM4, MFG-E8, protein S (ProS) and growth arrest-specific 6 (Gas6) of macrocytes which are necessary to carryout coupling and engulfing of apoptotic cells may also reduce the phagocytotic digestion of tumor DNA and other tumor-specific components of cancer cells, resulting in increased release into the bloodstream. The present disclosure is also directed to oral dosage medications for briefly enhancing ctDNA presence for liquid biopsy sensitivity enhancement, comprising effective dosages of at least 3 different tumor cell apoptosis enhancers of different apoptosis pathways, and an inhibitor of phagocytotic digestion of apoptosing cells. The dosage release of the components should best be timed to reduce phagocytic digestion of apoptosing cancer cells induced by apoptosis enhancers, to increase the presence and concentration of ctDNA in the screening patient's bloodstream at a predetermined liquid biopsy time.

Further in accordance with various aspects of the present disclosure, asymptomatic patient cancer screening methods can preferably comprise pre-liquid biopsy sensitivity optimization steps including brief fasting or anti-apoptosis treatment to briefly limit apoptosis to facilitate later brief enhancement of the amount of cancer cell apoptosis optimized for liquid biopsy. The sensitivity optimization may also comprise brief exocellular DNAse I inhibition and/or brief reduction of phagocyte digestion of DNA from apoptotic cells, to enable bloodstream concentration increase of ctDNA for liquid biopsy. An important aspect of liquid biopsy sensitivity enhancement is brief enhancement and restoration of tumor cell apoptosis function. Cancer cells typically have one or more natural apoptotic pathways disabled or compromised, which would otherwise normally result in their natural death and removal. Particularly preferred is brief multiple apoptotic system enhancement, timed to optimize ctDNA concentration peaking in the bloodstream. The screening methods further comprise collecting a liquid biopsy sample from the screening patient, timed with maximized ctDNA concentration in the screening patient's bloodstream. The liquid biopsy sample can be collected in a variety of ways, for example from plasma, urine, and/or saliva. The DNA including ctDNA collected from the liquid biopsy sample may be stabilized. The liquid biopsy sample is then tested to detect the presence of ctDNA. If ctDNA is not detected, the screening process may be (successfully) completed. If ctDNA is detected, it may be quantified, and further analyzed to obtain diagnostic information. In addition, ctHistone content of the liquid biopsy sample may be detected, quantified and analyzed for diagnostic information. Similarly, Cytochrome c, and other cancer markers which may be present in the liquid biopsy sample, may be analyzed for diagnostic information. The various analyses may be combined to improve the diagnosis and characterization of tumor cells which shed the ctDNA detected in the screening patient's liquid biopsy sample.

As described, the concentration of ctDNA in the bloodstream of a patient with even a small undiagnosed tumor mass, may be increased prior to liquid biopsy or other sampling, by means for increasing apoptosis of malignant cells, and by brief fasting from apoptotic inducers prior to the test regimen. In addition, the amount and concentration of ctDNA from malignant cells may be briefly increased for liquid biopsy sampling by reducing the phagocytic digestion of apoptotic tumor cells, and/or management (temporary reduction) of the rate of degradation of ctDNA present in the patient's bloodstream. The concentration and amount of DNA in a screening patient's circulating bloodstream is a function of the rate and amount of release of DNA from cells, and the rate of degradation of DNA which has been released into the bloodstream. ctDNA may normally have a limited half-life, for example, of only an hour or two in the bloodstream, which limits the detectable concentration and amount of ctDNA which is recovered in a sample volume of blood or other body fluid. DNA, including cfDNA, and ctDNA if present, is normally degraded in the bloodstream by endonucleases such as deoxyribonucleases (DNases). The human glycoprotein DNase I family comprises, inter alia, DNase I, DNase X, DNase γ DNAS1L2 and DNAse1L3, of which DNase I, DNAse1L2 and DNAse1L3 are secreted extracellularly. DNAse I (including DNase 1L3) is the major nuclease present in blood (and corresponding patient plasma, serum, urine and saliva samples) which hydrolyses ctDNA and functions to clear serum of DNA, including ctDNA. Further in accordance with the present disclosure, detection of ctDNA from a screening patient's malignant cells (if present) may be improved and enhanced by administration of means for inhibiting degradation of ctDNA in the patient's bloodstream prior to test sampling of body fluid. A variety of DNAse inhibitors are known, and others will be developed. DNase I inhibitors comprise curcumin (including its derivatives), somatostatin which decreases serum DNase I in a dose dependent manner by down regulation of DNase I gene expression, and EDTA which functions by complexing Ca and Mg ions. A low molecular weight compound from M. echinospora, MG299-fF35, inhibits DNase I by direct binding to the enzyme. A specific inhibitor of DNase I is also known from N. tabacum cell cultures. Antibiotics isolated Streptomyces (actinomycin D, nogalamycin, daunomycin, neomycin B and paromomycin interact with DNA to inhibit DNase I binding which inhibits degradation. By inhibiting degradation of ctDNA in the bloodstream, while ctDNA is being released in the bloodstream by apoptosis of malignant cells, the concentration and amount of ctDNA is increased for detection and quantitation. By coordinating brief increase in apoptotic release of ctDNA into the bloodstream, while inhibiting degradation of ctDNA in the bloodstream, the concentration and amount of ctDNA is increased for detection in the screening patient's blood and other body fluids, in accordance with the present disclosure.

After or upon withdrawal or other collection of a bodily fluid sample from the patient, DNA in the sample, including ctDNA if present, may conventionally be preserved by temperature reduction, and treatment with EDTA which complexes cations necessary for DNAses to hydrolyse the DNA present in the sample, adsorption on a stabilizer and/or other processes.

By “detection” of ctDNA is meant the identification of the presence of ctDNA in the screening patient's fluid sample. The detection of ctDNA is an indication of the presence of tumor cells in the patient's body. By “quantification” of ctDNA is meant the determination of the amount of ctDNA present in the screening patient's fluid sample or specimen. The amount of ctDNA is an indication of the size and/or cell-shedding volume of tumor cells in the patient's body. By “analysis” of ctDNA is meant the determination of the specific type, types, genetic locations of mutation(s) of the ctDNA present in the screening patient's fluid sample or specimen. The specific types of genetic DNA mutations are an indication of the specific type of tumor(s) and their tissue(s) of origin in the patient's body, and can be useful in selecting treatment regimen(s).

Cancer cells generally have disabled programmed cell death apoptosis functions, which protect the normal genome. Programmed cell death of cancer cells with mutated genomes is normally dependent on genetically encoded apoptotic signals or activities within the (dying) cell. Apoptosis is an active, programmed process of autonomous cellular disassembly, fragmentation and deconstruction of the dying cell, while limiting immunological inflammation. Effector caspase-3, -6, and -7 typically carry out the cell deconstruction. Such ctDNA concentration enhancement can be produced by inducing a substantially general or generic cancer-cell-killing process, such as restoring defective cancer-cell apoptosis, to briefly stimulate apoptosis in a broad range of cancer types. The brief apoptosis reactivation of defective cancer cell apoptosis should best be carried out without substantial permanent toxicity to normal, nonmalignant screening patient cells. The briefly increased circulating tumor DNA from restored/reactivated apoptosis induction facilitates early detection of smaller, earlier tumors which may be present in the screening patient's body, thereby permitting earlier treatment with potentially improved outcomes. The reactivated apoptosis induction does not need to be effective cancer treatment, and can be relatively safe for screening purposes separate and distinct from harsh treatment regimens which can adversely also significantly harm normal cells. Such embodiments of the present methods can be especially useful for broad cancer screening for wide ranges of different types of human cancers, because they can safely and briefly increase the ctDNA concentration in the body fluids of screening patients by increasing apoptosis and/or (less preferably) necroptosis to increase the rate of shedding of ctDNA, malignancy-marker RNA and/or malignancy marker proteins into the bloodstream or other body fluids, from even a relatively small, early stage tumor.

The present disclosure is also directed to screening methods for detecting the presence of cancer cells in a patient's body, comprising the steps of enhancing apoptosis of cancer cells within the patient, while simultaneously reducing the phagocytic digestion of apoptotic cells to increase the amount of DNA from apoptotic cancer cells which becomes circulating tumor DNA, obtaining a liquid biopsy sample while ctDNA concentration is enhanced by enhanced cancer cell apoptosis and reduced phagocytotic digestion of apoptotic cells, and determining the presence or absence of ctDNA in the liquid biopsy sample.

In addition, aspects of some methods herein may comprise collection and analysis of cancer cell histones, preferably those which are bound to or associated with ctDNA. In normal and cancer cells, DNA is wound around histone protein structures. Cell apoptosis can partially hydrolyse the cell DNA, typically for ctDNA to fragments in the range of about 50 to about 150 bp. When cells undergo apoptosis to release (partially hydrolysed) DNA fragments as cfDNA (including ctDNA if present) into the bloodstream and other fluids, the DNA fragments can remain partially attached or otherwise associated with histone components, which can partially protect the cfDNA from more rapid degradation by DNAses. Nucleosomes comprise complexes of histones and DNA which are released from cells into the blood circulation upon cell death. In cancer cells the histone proteins of cancer cells associated with ctDNA can also typically be abnormal, such as by having post-translational modification of histone tails including tumor-type-specific aberrant patterns of histone methylation and acetylation. Histones from patient fluid (e.g., serum) and other tissue samples are conventionally detected and analyzed; specific patterns of acetylation and methylation markers of histones are conventionally associated with, and are defined for, specific types (and tissue location) of cancers. In this regard, genome-wide mapping of malignant cells has defined chromatin changes which develop in tumors. Decades of research have compiled a library of standardized histone PTMs which are known to be altered in cancer and are conventionally known as histone onco-modifications. Cancer cells can have diminished histone acetylation and methylation, for example at acetylated Lys16 and trimethylated Lys20 residues of histone H4. The loss of histone H4 lysine 16 acetylation (H4K16Ac) and lysine 20 trimethylation (H4K20Me3) are characteristic of, and statistically diagnostic for, many human cancers. For example, global H3 and H4 hypo-acetylation statistically correlate with cancer cell phenotype in breast and prostate cancers. Accordingly, histone analysis is usefully diagnostic for malignancy. Histone fragments circulating in the blood (or other patient fluids) are referred to herein as cfHistones, while abnormal cancer cell Histones circulating in the bloodstream are referred to herein as ctHistones. It is hypothesized that the affinity of ctDNA for gold surfaces, such as gold nanoparticle surfaces which facilitates colorimetric detection of ctDNA, may also be coincident with affinity of ctDNA-ctHistone complexes.

In accordance with additional aspects of the present disclosure, patient liquid biopsy samples which have detected ctDNA may further be analyzed for aberrant ctHistone. Circulating DNA can also comprise DNA fragments bound to histone components such as H3K27me2, which can provide limited protection or delay from digestion by DNAse in the bloodstream, as well as a source of ctHistones for diagnostic assay as described herein.

Accordingly, in one aspect, provided herein is a method for detecting cancer in an asymptotic human patient not previously diagnosed with cancer, comprising the steps of: administering a short-term effective dose to the patient of a drug means for selectively inducing apoptosis and/or ferroptosis in human cancer cells; discontinuing the administration of drug means for selectively inducing apoptosis and/or necroptosis in human cancer cells within 3 days of the initial administration; obtaining a sample of a body fluid from the patient within 7 days of the initial administration of the apoptosis inducing drug means; and analyzing the sample fluid from the patient for the presence of circulating tumor DNA and/or cancer-characteristic circulating biomarkers.

Other objects and features will be in part apparent and in part pointed out hereinafter.

### DETAILED DESCRIPTION

Provided herein is a method for detecting cancer in an asymptotic human patient not previously diagnosed with cancer. The method may comprise one or more of the following steps: (1) administering a short-term effective dose to the patient of a drug means for selectively inducing apoptosis and/or ferroptosis in human cancer cells; (2) discontinuing the administration of drug means for selectively inducing apoptosis and/or necroptosis in human cancer cells within 3 days of the initial administration; (3) obtaining a sample of a body fluid from the patient within 7 days of the initial administration of the apoptosis inducing drug means; and (4) analyzing the sample fluid from the patient for the presence of circulating tumor DNA and/or cancer-characteristic circulating biomarkers. These and other aspects of the present disclosure are discussed in further detail below.

As people age and mature, their cells are continuously subject to mutation from genetic, epigenetic and environmental factors and influences such as UV and ionizing radiation, environmental toxins, and/or imperfect replication, repair or transcription. Over time, progressively genetically or epigenetically mutated, or modified cells or mosaics of such cells can form, which can eventually fully progress to malignancy. To counter these effects, well-regulated cells have a variety of important mechanisms and pathways for DNA repair, oncogene suppression, removal of aberrant components by autophagy or exosome discharge, and ultimately programmed cell death (PCD) for mutated unregulated cells by apoptosis or necroptosis pathways. However, the various regulation-protective programmed cell death pathways themselves can also become downregulated, mutated, limited, silenced, disabled or otherwise functionally deactivated by mutations or other defects such as epigenetic hypermethylation and unregulated histone acetylation/deacetylation. Tumor suppressor genes such as p53, p16, p73, DLC1, PPARG, ST7, FOXO6, TET2, CYP1B1, SALL4, and DDIT3 can be epigenetically downregulated or silenced in tumors, and pro-apoptotic genes such as RASL11B, RASD1, GNG3, BAD, and BIK can be also be downregulated or inactivated. The PTEN gene encodes a phosphatase and tensin protein which is mutated in many cancers. The phosphatidylinositol-3,4,5-trisphosphate 3-phosphatase protein of the PTEN gene reduces levels of phosphatidylinositol-3,4,5-trisphosphate, and functions as a tumor suppressor by negatively regulating the Akt/PKB signaling pathway. The p16 protein (also known as p16^{INK4a} cyclin-dependent kinase inhibitor 2A, and multiple tumor suppressor 1), is a tumor suppressor protein encoded by the CDKN2A gene (also called the p16 gene). The p16 gene and its expressed protein act as a tumor suppressor which inhibits progression from G1 phase to S phase. p16 refers to expressed 16 kDalton protein molecular weight, while p16^{INK4} additionally refers to its inhibition of CDK4.

Screening patient cells also have self-protective mechanisms such as autophagy and oncogene expression, which can weigh against apoptosis, and which malignant cells can adapt to prevent their programmed cell death. Because there are a plurality of different programmed cell death pathways, dangerously aberrant cells with one or more still-functional programmed cell death pathways can continue to be eliminated by such alternate, remaining apoptosis pathway(s). However, cells which are sufficiently disabled so than none of the programmed cell death pathways are sufficiently active or effective to eliminate unregulated cells, can survive, multiply, spread and metastasize as uncontrolled malignant cancers.

Because genome-protective apoptosis can be initiated by multiple alternative pathways, functional downregulation or other deactivation of multiple, or substantially all pathways is typically present in malignant cancer cells. For purposes of asymptomatic general cancer screening sensitivity enhancement, it is only desirable to briefly increase the rate of apoptosis and concomitant ctDNA release from a portion of a patient's mutated cells with deactivated PCD pathways, rather than attempt a “cure” by killing all mutated or malignant cells in asymptomatic screening test patients. Screened patients identified as being at significantly increased risk by such enhanced screening testing, may be further tested and/or treated as appropriate.

Cancer cells have typically downregulated, overcome or otherwise disabled substantially all programmed cell death mechanisms which protect the stability of the human genome. Partially-mutated pre-cancer cells can continue to mutate, such as by hypermethylation of nuclear and mitochondrial DNA, or interference with normal histone acetylation, to further disable the protection of the genome. Those further mutated cells which survive mutation with more fully disabled programmed cell death mechanisms can proceed to malignancy. Fortunately, some apoptosis/necroptosis can be at least briefly partially restored in many types of cancer cells. Because loss of p53, p16 and/or other apoptotic/necroptotic functions are characteristic of most types of cancer, which permits them to survive, brief restoration of apoptotic or necroptotic function to a screening patient's undiagnosed cancer cells can at least briefly increase both cancer cell death and the consequent concentration of circulating tumor DNA and/or other abnormal cfNAs/RNAS and proteins in the patient's body fluid for purposes of screening detection. For example, inhibition of Pyruvate Dehydrogenase Kinases in cancer cells can reduce aerobic glycolysis, reduce lactate concentration, increase mitochondrial oxidation, decrease HIF1a expression, and importantly, re-activate caspase-mediated and p53 apoptosis function for inducing cancer cell death. Disabling of apoptosis is relatively generic to cancer cells; brief or short-term re-activation of apoptosis can at least briefly relatively generically kill some cancer cells, and increase the ctDNA concentration of a relatively generic, wide variety of undiagnosed early-stage cancers in a cancer-screening population.

In accordance with the present disclosure, cancer screening methods are provided in which multiple programmed cell death pathways are substantially simultaneously upregulated, repaired, or otherwise reactivated in order to restore genome protective mechanisms to sensitize or induce death of malignant cells which trigger one or more of the normal programmed cell death pathways. In accordance with preferred aspects of the present disclosure, brief administration of effective amounts of means for reactivation of apoptosis in asymptomatic patient cancer cells may comprise means for reactivating multiple, preferably at least 3 and more preferably at least 4, apoptotic pathways, most or all of which are downregulated, or otherwise functionally deactivated or silenced in malignant cancer cells. Many traditional natural compounds and foods with long-established safety experience can relatively safely enhance apoptosis, and accordingly are among the preferred apoptosis-inducing means for use herein.

Means for Reactivating Apoptosis Pathways and their Mechanisms in Cancer Cells

Useful means for reactivating various apoptosis pathways and their mechanisms in cancer cells include:

p53 means for activating p53-induced apoptosis in cancer cells.

PDK inhibition means for inhibiting pyruvate dehydrogenase kinase to decrease HIF1a expression and reactivate p53- and caspase-mediated apoptosis for inducing cancer cell death. Examples of such PDK inhibition means include dichloroacetate, oxamate, diisopropylamine dichloroacetate, and PDK1 ssRNA.

Bax activation means for activating expression of proapoptotic protein Bax in cancer cells. Bilobetin and isoginkgetin are examples of Bax activation means, as well as T. vulgaris phytochemicals.

Bcl-2 inhibition means for inhibiting cancer cell expression of anti-apoptotic protein Bcl-2. Examples of such Bcl-2 inactivation means include bilobetin, and mycoepoxydiene, a natural polyketide isolated from the marine fungal strain Diaporthe sp. HLY-1 associated with mangroves, piperlongumine, a natural alkaloid present in Piper longum Linn. Such Bcl-2 inhibition means may also typically inhibit expression of other antiapoptotic proteins. Piperlongumine can suppress activity of Akt/NF-kB, c-Myc and cyclin D1, and induce a mitochondrial apoptotic pathway by downregulating Bcl-2 levels. Cardanol monoene, a major phenolic component extracted from cashew nut shell liquid can reduce mitochondrial membrane potential (ΔΨm), upregulate expression of p53, increase cytosol cytochrome c, cleaved-caspase-3, and cleaved-PARP, and increase the Bax/Bcl-2 ratio to promote apoptosis.

Means for inhibiting the pI3K/Akt/mTOR signaling pathway. Panobinostat, piperlongumine, myricetin, caffeine (with antiautophagy chloroquine) are examples of PI3K/Akt/mTOR inhibitors.

Means for increasing the Bax:Blc-2 cancer cell ratio. Examples can include means Bax activation means and Bcl-2 inhibition means. S-Adenosyl-1-methionine (available as a non-prescription dietary supplement in the United States) is a naturally and widely occurring sulfonium compound which can induce cancer cell apoptosis and autophagy, and function as a methyl donor for cancer cell DNA methylation. S-Adenosyl-1-methionine can induce apoptosis by a caspase-dependent mechanism together with increasing the Bax/Bcl-2 ratio, activating the unfolded protein response, and activating CHOP and JNK. S-Adenosyl-1-methionine-induced autophagy can be limited by the autophago-lysosome inhibitor chloroquine. S-Adenosyl-1-methionine can increase p53 expression and when combined with autophagy inhibitor chloroquine can synergistically re-activate apoptosis together with increased cleavage of caspase-6 and PARP, and inhibition of the activity of the Akt/mTOR pathway. S-Adenosyl-1-methionine and/or its analogs in combination with chloroquine and/or its analogs is an effective means for briefly inducing cancer cell apoptosis for screening in accordance with the present disclosure. Mutually inhibitory crosstalk between autophagy and apoptosis can prevent apoptosis when autophagy signaling is upregulated by cancer mutation. This signaling crosstalk between autophagy and apoptosis pathways in cancer cells can accordingly be hijacked in mutated cells to favor autophagy and cancer cell survival rather than programmed cancer cell death. Autophagy upregulation permits cancer cells to survive, and to become resistant to anticancer agents. By administering autophagy inhibition means such as chloroquine, the apoptosis pathway blocked by upregulated autophagy crosstalk can be restored. Combining such means for inhibiting autophagy with apoptosis reactivating means such as S-Adenosyl-1-methionine is a preferred aspect of the present disclosure.

Caspase expression means for increasing cancer cell expression of cleaved caspase-3 and -7, up-regulating pro-apoptotic proteins Bad and Bax, and down-regulating anti-apoptotic proteins Bcl-xL and Bcl-2. Bayberry leaf flavonoids containing myricitrin (myricetin 3-O-rhamnoside) and quercetin (quercetin 3-rhamnoside) are examples of such caspase expression means; Myricetin can inhibit PI3K/Akt/mTOR signaling to induce apoptosis and procaspase-3 cleavage, and sulforaphane and myricetin can act synergistically therewith to induce apoptosis.

DNA demethylation means for reducing cancer cell DNA hypermethylation. Examples of such DNA demethylation means include compounds and agents such as SGI-110, Hinokitiol, a tropolone-related natural compound, can induce DNA demethylation by inhibiting the expression of DNMT1 and UHRF1 in cancer cells. The dichloromethane extract of S. atropatana is an example of means for reducing hypermethylation of p53 gene promoter and decreasing survivin mRNA. Green tea catechins such as epigallocatechin-gallate, alone and in combination with eugenol-amarogentin can achieve DNA hypomethylation through downregulation of DNMT1.

Nrf2-p53 means for upregulating Nrf2 expression by DNA demethylation and upregulating the interaction of Nrf2 with p53 is another example of such DNA demethylation means. Luteolin, a dietary flavone, is an example of such Nrf2-p53 means which inhibits the expression of DNA methyltransferases (transcription repressors), and increases the expression and activity of ten-eleven translocation (TET) DNA demethylases (transcription activators); luteolin can decrease methylation of the Nrf2 promoter region, increase mRNA expression of Nrf2, and increase TET1 binding to the Nrf2 promoter.

Histone demethylase inhibition means for inhibiting histone demethylase to restore functional cancer cell histone methylation for cancer cell apoptosis initiation. Lysine-specific histone demethylase 1A LSD1 inhibitor CBB3001 is an example of histone demethylase inhibition means.

Histone deacetylase inhibition means for inhibiting cancer cell histone deacetylase to initiate cancer cell apoptosis. Examples of such histone deacetylation inhibition means which can downregulate or otherwise inhibit histone deacetylase activity include compounds such as romidepsin and bromodomain inhibitors such as JQ1, vorinostat which targets HDAC1, HDAC2, HDAC3 and HDAC6.

Tumor suppressor upregulation means for upregulating at least 2 of tumor suppressor genes DLC1, PPARG, ST7, FOXO6, TET2, CYP1B1, SALL4, and DDIT3, together with upregulation of at least two of pro-apoptotic genes RASL11B, RASD1, GNG3, BAD, and BIK. Thymoquinone, the major biologically active compound of black seed oil, is a natural plant product example of such tumor suppressor upregulation means.

p73 apoptosis activating means for reactivating cancer cell p73 apoptosis. Thymoquinone is an example of such p73 apoptosis reactivation means. Thymoquinone can initiate programmed cancer cell death and alteration of the mitochondrial membrane potential, associated with re-activation of p73 expression and increase of caspase-3 cleaved subunits in an effective dose-dependent manner. Grape pomace extract can also activate cancer cell p73 expression.

PI3K/Akt inhibition means for downregulating the cancer cell PI3K/Akt pathway to decrease active histone mark H3K4me3. Magnolol is an example of such PI3K/Akt inhibition means.

Means for activating cancer-deregulated signaling pathways of WNT/β-catenin and janus kinase/signal transducers and activators of transcription (JAK-STAT) pathways. Apigenin can restore apoptosis in tumor necrosis factor-related apoptosis-inducing ligand (TRAIL)-resistant cancers.

Histone deacetylase inhibition means for inhibiting histone deacetylation which prevents cancer cell apoptosis. Various phenolics, polyketides, tetrapeptide, terpenoids, alkaloids, and hydoxamic acid such as components of natural and dietary origin including butein, protocatechuic aldehyde, kaempferol (e.g., from grapes, green tea, tomatoes, potatoes, and onions), resveratrol (e.g., from grapes, red wine, blueberries and peanuts), sinapinic acid (e.g., from wine and vinegar), diallyl disulfide (e.g., from garlic), and zerumbone (e.g., from ginger), as well as prescription drugs vorinostat, romidepsin, belinostat, and panobinostat are examples Histone deacetylase inhibition means. The plant isoquinoline alkaloid berberine is another example of histone deacetylation inhibitor means, which can downregulate oncogenes TNF-α, COX-2, MMP-2, and/or MMP-9, and upregulate p53 mRNA and protein expression, regulate Bcl-2/Bax proteins and/or reactivate the caspase cascade apoptotic pathway in cancer cells. Magnolol and polyphenol components derived from Magnolia officinalis are also examples natural class I histone deacetylase inhibition means, for reactivating cancer cell apoptosis, while enhancing pro-apoptotic TRAIL-R2 (protein death receptor pathway), Bax, caspase 3, cleaved caspase 3, and cleaved PARP.

Lysomotropic means for increasing the permeability of lysosomes and enhancing apoptosis. Examples of such lysomotropic means include loratadine (e.g., non prescription H1 antagonist, at sub-micromolar concentrations from 5 and 10 mg tablets/day), astemizole, ebastine, diphenhydramine and chlorpheniramine. Lysosomes are acidic cell organelles which can have an important pathway role in programmed cell death. Permeabilization of the lysosomal membrane and release of hydrolytic enzymes to the cytosol or as exosomes accompanies apoptosis in some programmed cell death pathways. Lysomotropic compounds, including many medications, with a basic component can become protonated and enriched within the acidic pH lysosomes, increasing their permeability and facilitating apoptosis. A variety of lysomotropic compounds are conventionally employed as antimalarial, antihistamine, antidepressant, antipsychotic agents which can increase cancer cell sensitivity to programmed cell death. As described, a common characteristic of cancer cells is reliance on inefficient energy pathways such as glycolysis and pentose phosphate shunt, compared with oxidative phosphorylation, which discharges acid from the cancer cell. Blocking proton currents in cancer cells by Hv1 ion channels can produce cancer cell acidification which can induce apoptosis. In addition, Diphenhydramine (an H1 histamine receptor antagonist and a relatively safe nonprescription drug), can impair the STAT3/MCL-1 cancer cell survival signaling pathway to induce apoptosis.

Means for inhibition of Pyruvate Dehydrogenase Kinases in cancer cells to reduce aerobic glycolysis. An example of such means for inhibition of Pyruvate Dehydrogenase Kinases is dichloroacetate. Tumor cells can typically increase aerobic glycolysis (the Warburg Effect) such as facilitated by overexpression of the oncogene Myc and hypoxia-inducible factor 1 alpha (HIF1a) for energy generation, in preference to mitochondrial oxidation. HIF1a upregulates the Pyruvate Dehydrogenase Kinases (PDK1, PDK2, PDK3, PDK4) plasma membrane glucose transporter GLUT1 and glycolysis enzymes. This can inhibit the Pyruvate Dehydrogenase Complex (PDC) and oxidative phosphorylation in the mitochondria, resulting in increased rates of glycolysis and lactate generation. Upregulated PDKs and high lactate concentration can stabilize HIF1a overexpression to maintain the Warburg metabolism for tumor survival, while diminishing pro-apoptotic factors from mitochondrial function which can inhibit p53, p16, p21, anoikis and other forms of apoptosis/necroptosis which could otherwise produce programmed death of the cancer cells. Warburg behavior and/or disabling of normal programmed cell death functions are typical or general to many malignancies. Means for inhibition of Pyruvate Dehydrogenase Kinases (PDK) include a variety of PDK inhibitors specifically including those which can act at pyruvate, nucleotide, lipoamide, and/or allosteric binding sites of the Pyruvate Dehydrogenase Kinases. Dichloroacetate is a simple, very inexpensive drug agent which has been used medically for decades to control glucose and fat metabolism. Dichloroacetate (DCA) is most active in inhibiting PDK2, is active against PDK1 and PDK4, and has lower activity in blocking PDK3. DCA is rapidly absorbed after administration to produce rapid stimulation of Pyruvate Dehydrogenase Complex activity within 30 minutes of oral or parenteral administration Repeated dosing can sustain Pyruvate Dehydrogenase Complex increase. Dichloroacetate has been considered safe when administered daily for one to two decades in childrens' primary mitochondrial diseases such as congenital PDC deficiency. It is typically well tolerated in adults, provided that dose adjustments are made for age and GSTZ1 haplotype. Other mono- or di-halogenated derivatives of short-chain fatty acids can activate the Pyruvate Dehydrogenase Complex by inhibiting Pyruvate Dehydrogenase Kinases.

Dysfunctional tumor cell p53 and other apoptosis systems can also be repaired, replaced or otherwise restored to functionality by CRISPR techniques, which can simultaneously probe and control several genetic circuits.

Ferroptosis stimulation means for stimulating ferroptosis in cancer cells

Apoptosis and/or Ferroptosis Stimulation or Reactivation Means

Preferably, the cancer cell apoptosis and/or ferroptosis stimulation or reactivation means administered prior to liquid biopsy will comprise effective amounts of at least 3, and more preferably at least 4 different chemical compounds selected from at least 3 and preferably at least 4 of the following:
PDK means for inhibition of Pyruvate Dehydrogenase Kinases,
DNA demethylation means for reducing cancer cell DNA hypermethylation,
Autophagy inhibition means for inhibiting cancer cell autophagy,
Nrf2-p53 means for upregulating Nrf2 expression by DNA demethylation,
p73 apoptosis activating means for reactivating cancer cell p73 apoptosis,
Histone deacetylase inhibition means for inhibiting histone deacetylation,
Lysomotropic means for increasing the permeability of lysosomes and enhancing apoptosis, and
Means for suppressing PI3K/Akt/mTOR signaling add ferroptosis definition means

As indicated, to survive and multiply, cancer cells containing mutated DNA must reduce or fully disable the various programmed cell death (e.g., apoptosis, ferroptosis) functions of normal cells. These normal apoptosis/ferroptosis genome-protecting functions are complex, so can be disabled by mutation at many parts of the apoptosis/ferroptosis pathways. Accordingly, it is preferred that the means for at least partially restoring apoptotic function to human cancer cells be capable of restoring multiple apoptotic pathways to be more inclusive of the types of cancer cells detectable by screening of asymptomatic patients in accordance with the present disclosure. One major apoptosis pathway involves the human tumor protein p53, also known as p53, phosphoprotein p53, tumor suppressor p53, antigen NY-CO-13, or transformation-related protein 53 (TRP53). p53 is a protein produced by normal human cells. The p53 system, known as “the guardian of the genome”, acts to prevent genome mutation by killing aberrant cells by the mechanism of apoptosis and related functions. Most human cancer cells are characterized by dysfunctional or disabled human cell p53, which permits them to proliferate, when they would otherwise be subjected to programmed cell death. Restoration of disabled apoptosis is one goal of cancer treatment. In addition to p53, a variety of other cell mechanisms also protect against malignant cell development into dangerous tumors. After p53, p16 may be the second most common tumor suppressor gene, with the frequency of p16 inactivation in cancers ranging from about 20% in breast cancer to about 85% in pancreatic adenocarcinoma. p16 DNA methylation is known to be one of the most frequent events in cancer development. The p16 gene belongs to INK4 family of genes and is made up of four members: p16 INK4A, p15 INK4B, p18 INK4C and p19 NK4D, which inhibit cell growth and suppress tumours. Reactivation of p16 tumor suppression function can initiate cancer cell apoptosis. As indicated, the p21 system also can sometimes inhibit the continuation of malignant cells. The PTEN tumor suppressor is frequently mutated in human tumors. Loss of PTEN is associated with constitutive survival signaling through the PI3K/AKT/mTOR pathway. Several therapeutic treatments have been explored to restore APC function including targeting APC mutant cells for apoptosis. Undiagnosed and undetected cancer cells with misfolded p53 in screening patients could be induced to initiate apoptosis by reversing the p53 misfolding. Briefly reactivating and/or stimulating multiple aspects of normal cell apoptosis mechanisms which can be disabled in malignant cells, can broaden the range and types of cancer cells which can release their mutated ctDNA and other mutated RNA and proteins upon programmed apoptotic malignant cell death to be detected by ctDNA screening of asymptomatic patients.

In accordance with one aspect of the present disclosure, brief enhancement of p53 and/or other apoptotic programmed cell death functions is used to briefly increase the concentration of ctDNA in screening patient bodily fluid concentration to enhance testing detection. Cancer cells similarly can avoid destruction by inhibiting necroptosis. Means for inducing necroptosis such as inositol phosphates can trigger an “executioner domain” of a cancer cell's MLKL molecules to break down the integrity of the cell membrane and kill the cell. Necroptosis inducing means may also be administered to screening patients, alone or in combination with means for inducing apoptosis, on a short-term basis to briefly increase ctDNA concentration within or over a period of several days to detect cancer at an early stage as described herein. Apoptosis/ferroptosis is more fragmenting and proteolytic and rapid than necroptosis and less immunostimulative, so is preferred for screening.

Dysfunction of p53, which protects against genetic mutations including cancer, is substantially common in human malignancy, so briefly restoring or otherwise enhancing p53 function to briefly increase ctDNA in body fluids such as blood samples for liquid biopsy is useful for broad cancer screening, especially for early cancer detection in patients not previously diagnosed with cancer. A wide variety of brief administrations of p53 and/or other apoptosis reactivators can produce short-term increases in ctDNA concentration in the blood of screening patients who have undetected early-stage cancer.

Many anti-cancer drugs are adapted for treatment of specific types of cancer malignancies derived from specific tissues of origin. However, upregulated glycolysis is characteristic in wide range of human cancers. Drugs such as dichloroacetate (DCA), oxamate, and PDK1 ssRNA can inhibit pyruvate dehydrogenase kinase, to reactivate mitochondrial function and decrease glycolytic pathways in wide ranges of different types of tumor cells, to increase cell cycle arrest and apoptosis. Dichoroacetate itself is rapidly absorbed into mitochondria (of cancer cells) and readily passes through the blood-brain barrier. Mimosine, mycophenolic acid, baicalein, baicalin, wogonin and fisetin plant flavonoids, and ellagic acid can promote apoptosis and increase the activity of caspases. A wide variety of other natural and synthetic drug agent means for inducing apoptosis and/or necroptosis of cancer cells are also known, and are summarized in Table 1 below.

TABLE 1
Dichloroacetate (e.g., sodium salt)
Diisopropylamine dichloroacetate (DADA) is a relatively safe inhibitor of PDK4 (pyruvate
dehydrogenase kinase 4). DADA is the active component of pangamic acid which has been
used for many years for treatment of chronic liver disease.
Os(II) complex 2 [Os(η⁶-pcym)(bphen)(dca)]pf₆ (pcym = p-cymene, bphen =
bathophenanthroline, and dca = dichloroacetate)
Pyruvates such as 3-bromopyruvate
2-deoxyglucose
R-lipoic acid
2-chloroproprionate
Inositol esters or ionic complexes (e.g., inositol hexa (N-methylnicotinate-dichloroacetate) and
tetra (dichloroacetyl) gluconate (potassium salt), Potassium tetra (dichloroacetyl) glucuronate,
Inositol hexa (N-methynlicotinatedichloroacetate
(R)-3.3.3-trifluoro-2-hydroxy-2-methyl propionamides, eg, Nov3r (Novartis)
Anilide tertiary carbinols, e.g., AZD7545 (AstraZeneca) inhibits PDK1-3
N-(2-aminoethyl)-2{3-chloro-4-[(4isopropylbenzyl)oxy] phenyl}acetamide; Pfz3 (Pfizer)
4,5-diarylisoxazoles
VER-246608 Pan-PDK isoform inhibitor at ATP binding site
Radicicol (monorden)
Mitaplatin
Hemoglobin-DCA conjugate
Mito-DCA
Phenylbutyrate
Betulin is a natural plant triterpene which can trigger apoptosis via caspase-3 and caspase-9
pathways. Betulinic acid, including ester derivatives, can be synergistic in combination with
DCA
DCA-oxaliplatin derivatives, e.g., fusion molecule of 1 platin: 2DCAs via ester linkages;
Honokiol DCA (ester derivative from Magnolia grandiflora)
Pyruvate analogs containing phosphonate or phosphonate group (e.g., acetyl phosphinate)
CPI-613, lipoate derivative which inhibits lipoate interactions
M77976 (dihydroxyphenyl pyrazole derivative) binds to PDK4 ATP pocket
Aromatic DCA derivatives bind to PDK1 ATP pocket
DCA-loaded tertiary amines, reported to increase DCA stability.
Furan and thiophene carboxylic acids, which are allosteric pyruvate site binding PDK2
inhibitors
Benzodiazepinediones
Spiro-oxindoles
Actinomycin D in combinations with other apoptosis/necrotosis enhancers
inositol phosphates for inducing necroptosis
drug means for inhibiting p53 MDM2-MDM4 interaction. Examples include RG7112,
RG7388, MI-77301, AMG232, MK-8242, CGM097, DS-3032b, RO6839921, stapled alpha-
helical peptide ALRN-6924 (Aileron Therapeutics) (NCT 02264613), HPV E6 (e.g., small-
molecule inhibitors of the p53-MDM2 protein interaction such as the nutlins (e.g., nutlin 3a
which is a competitive inhibitor of MDM binding to p53)
Agents which deplete mutant p53 such as Ganetespib, Onalespib, Luminespib
gene replacement means for reintroduction of apoptotically functional p53 genes into the
patient's cancer cells (“transplantation”) such as Gendicine (Ad-53) can induce apoptosis to
increase ctDNA concentration
p53 restorative low molecular weight compounds and peptides such PhiKan083, MIRA-1,
STIMA-1, ZMC1, PK7088, PK11000, PK11007, PK11011, PRIMA-1 and APR-246/PRIMA-
1 (pro-drugs that convert to methylene quinuclidinone for binding to p53), ReAcp53, and
pCAP-250 can reactivate mutant p53 and restore its transcription activity
Normal-type p53, delivered by adenovirus vectors (such as Gendicine, Advexin) can increase
ctDNA after administration, for screening enhancement
Oncolytic viruses designed to selectively kill p53 defective cells
siRNA and antisense RNAs which inhibit negative regulators MDM2, MDM4, MDMX
Navitoclax, 4-(4-{[2-(4-Chlorophenyl)-5,5-dimethyl-1-cyclohexen-1-yl]methyl}-1-
piperazinyl)-N-[(4-{[(2R)-4-(4-morpholinyl)-1-(phenylsulfanyl)-2-butanyl]amino}-3-
[(trifluoromethyl)sulfonyl]phenyl)sulfonyl]benzamide can induce apoptosis in senescent cells
but reduces red blood cell lifespan so is dose-limited for extended cancer treatment purposes.
Dasatinib (a broad-spectrum kinase inhibitor)
Myricetin and structurally similar fisetin, luteolin, and quercetin as members of the flavonol
class of flavonoids and related compounds including their precursors and derivatives, can
promote cancer cell apoptosis. Quercetin is a well-known relatively safe plant flavonoid which
can induce apoptosis of cancer cells without substantially affecting non-cancer cells.
Rapamycin and its analogs can partially substitute for p53 dysfunction.
Perifosine (1,1-Dimethylpiperidinium-4-yl octadecyl phosphate), an Akt inhibitor, can be
safely administered for ctDNA enhancement, but it lacks sufficient anti-cancer efficacy for
cancer therapy.
Dichloroacetate derivatives such as Adenosine Triphosphate Competitors Targeting Pyruvate
Dehydrogenase Kinase, eg 2,2-Dichloro-N-(4,6-dichloro-2-methylpyrimidin-5-yl)acetamide;
2,2-Dichloro-N-(4,6-dichloropyrimidin-5-yl)acetamide: 2,2-Dichloro-N-(4-chloro-3-
(trifluoromethyl)phenyl)-Acetamide
Dichloroacetophenone (DAP) can increased apoptosis by inhibiting PDK1
Aplidene, abietane, natural plant compounds (and their derivatives) can induce apoptosis in
cancer cells
Tanshinone hydrophobic abietane diterpenes present in miltiorrhiza Bunge roots, can induce
both early and late apoptosis, decrease expression of the anti-apoptotic protein Bcl-2, Bcl-xl
and increase expression of pro-apoptotic protein Bax, increase expression levels of PTEN, and
reduce the phosphorylated levels of Akt (protein kinase B)
The conventional antibiotic, clofoctol, can induce apoptosis of cancer cells (e.g., gliomas).
p53 reactivators PRIMA-1, strylquinazoline CP-31398, and CDB3
Epigallocatechin-3-gallate can induce apoptosis via demethylation and reactivation of the p16
gene. EGCG can inhibit NF-kB, and interact with DNA methyltransferases and histone
deacetylases.
Cardiovascular Drugs Hydralazine and Procainamide as demethylation agents
Melatonin (an endogenous indoleamine produced by the pineal gland) is a safe, very
inexpensive, non-prescription supplement which can enhance or otherwise reactivate cancer
cell apoptosis, even while having antiapoptotic function in normal cells.
Magnolol, a natural compound constituent of Magnolia officinalis, can reactivate disabled
apoptosis in cancer cells by downregulating the PI3K/Akt pathway, eg to decrease the active
histone mark H3K4me3.
Chinese bayberry leaf flavonoids can induce apoptosis in cancer cells via the Erk pathway.
Chinese bayberry myricitrin 3-O-rhamnoside and quercetin 3-rhamnoside can increase
expression of cleaved caspase-3 and -7 and induce apoptosis via an Erk-dependent caspase-9
activation intrinsic apoptotic pathway by up-regulating the pro-apoptotic proteins (Bad and
Bax) and down-regulating the anti-apoptotic proteins (Bcl-xL and Bcl-2).
nummularic acid, a major chemical constituent of Fraxinus xanthoxyloides, a medicinal plant
long used in traditional medicine can increase cancer cell apoptosis, activate adenosine
monophosphate-activated protein kinase (AMPK), while increasing acetyl CoA carboxylase
phosphorylation and decreasing pS6 phosphorylation, the two major substrates of AMPK.
Apigenin, a plant-derived flavonoid, can restore apoptosis in tumor necrosis factor-related
apoptosis-inducing ligand (TRAIL)-resistant cancers; it has higher effects when used in
combination with miR-423-5p inhibitors or miR-138 mimics.
Dichloromethane extracts of Scrophularia atropatana reactivate hypermethylated p53
promoter, to increase p53 gene expression, decrease survivin (an oncogene under functional
p53 control) mRNA, and promote apoptosis of cancer cells.
T. vulgaris plant components can increase Bax expression and decrease in the trimethylation
of the lysine 4 on the histone H3 protein (H3K4me3)
epigallocatechin gallate, eugenol clove component, and amarogentin component of chirata
plant alone or in combination can also reduce expression of DNA methyltransferase 1 to
induce promoter hypomethylation of LimD1 and P16 genes.
Crocin, derived from dried stigmas of Crocus sativus L. (saffron), is a natural product which
can induce cancer cell apoptosis.
Curcumin, a natural component of turmeric from Curcuma longa rhizomes, can promote
apoptosis, inhibit the JNK pathway, and/or repress trimethylation of the lysine 4 on the histone
H3 protein (H3K4me3). Curcumin can also inhibit DFF40/CAD to limit DNA fragmentation
during apoptosis, but does not prevent apoptotic cell death and induces apoptosis in gastric
carcinoma AGS cells and colon carcinoma HT-29 cells through mitochondrial dysfunction and
endoplasmic reticulum stress. Curcumin and other turmeric components can be readily
degraded and have relatively low oral bioavailability. Accordingly, a variety of curcumin
derivatives and other turmeric formulations with enhanced bioavailability such as curcumin
analog diarylheptanoids and diarylpentanoid monocarbonyl derivatives are available, and
others are being developed.
Grape pomace extract comprising physiologically active phenolic compounds is a natural plant
product can increase cancer cell apoptosis.
Hinokitiol, a tropolone-related natural compound, can induce cancer cell apoptosis.
Berberine, a natural plant isoquinoline alkaloid non-prescription supplement, can reactivate
cancer cell apoptosis.
Delphinidin, a major anthocyanidin compound found in various fruits, can induce p53-
mediated apoptosis in cancer cells by suppressing HDAC activity and activating p53
acetylation
Sulphoraphane (1-isothiocyanato-4-(methylsulfinyl)-butane) can restore apoptosis by a variety
of mechanisms
Mycoepoxydiene, a polyketide isolated from the marine fungal strain Diaporthe sp. HLY-1
associated with mangroves, can induce apoptosis and inhibit the expression of anti-apoptotic
proteins such as Bcl-XL and Bcl-2, two targets of NF-κB.
Kaempferol and 5-FU can upregulate the expression levels of Bax and downregulate the
expression levels of Bcl-2 and thymidylate synthase in cancer cells
Troline can induce apoptosis and suppress autophagy
Chloroquine, a safe over-the-counter compound (including its derivative hydroxychloroquine)
is a proaptotic agent which can increase cancer cell Bax:Bcl2 ratio to enhance apoptosis over
cancer cell survival mechanisms, and reactivate cancer cell apoptosis. Chloroquine and
hydroxychloroquine are lysosomotropic agents which disrupt lysosomal fusion degradation
and obstruct lysomally-derived nutrient supply to cancer cells
Caffeine (1,3,7-trimethylxanthine) a commonly consumed food ingredient, can induce
apoptosis
Antihistamines diphenhydramine and chlorpheniramine can enhance apoptosis
Anthocyanin-rich plant phenolic extracts such as cyanidin-3-O-glucoside of red and purple
grapes, purple sweet potato, purple carrot, black and purple beans, black lentil, black peanut,
sorghum, black rice, and blue wheat can induce apoptosis and decrease expression of anti-
apoptotic proteins cIAP-2, XIAP and survivin.
Enterodiol transformed by human intestinal bacteria from lignans contained in various whole-
grain cereals, nuts, legumes, flaxseed, and vegetables can increase cancer cell apoptosis.
Various compounds of dietary nut plants including peanut (actually a legume), cashew
(Cardanol monoene), hazelnut, pistachio, walnut (phytomelatonin+), pecan and the like are
proapoptotic.
Histone deacetylase inhibitors sodium butyrate and panobinostat can activate
intrinsic/extrinsic apoptotic pathways and inhibit the AKT-mTOR pathway
Cationic amphiphilic antihistamines such as Loratadine (e.g., non prescription H1 antagonist,
at sub-micromolar concentrations from 5 and 10 mg tablets/day), astemizole and ebastine can
function as inducers of lysosomal cancer cell death
The fruit of Ginkgo biloba L. a traditional Chinese medicine, can increase release of
Cytochrome C from mitochondria to cell cytosol and induce cancer cell apoptosis.
The common food plant spice oregano can decrease antiapoptotic Bcl-2, VEGFR-2, CD24,
and EpCAM expression while increasing proaptotic caspase-3 expression in cancer cells
Imatinib, a 2-phenyl amino pyrimidine derivative that inhibits tyrosine kinase enzymes, with a
specific target of the TK domain in abl (the Abelson proto-oncogene), thereby limiting the
PI/PI3K/AKT/BCL-2 pathway which cancer cells can use to protect against cell death by
apoptosis.
Extracts (including analogs) from medicinal plants Ammania baccifera, Asclepias curassavica,
Azadarichta indica, Butea monosperma, Croton tiglium, Hedera nepalensis, Jatropha curcas,
Momordica charantia, Moringa oleifera, Psidium guajava, are reported to have
antiproliferative, pro-apoptotic, anti-metastatic and anti-angiogenic activity with minimum or
negligible toxicity.
Galangin is a natural flavonoid which is an active ingredient in galangal, a spice also used in
traditional Chinese medicine, reported to be non-toxic to humans but toxic to tumor cells.
Galangin can activate caspase-3, down-regulate Bcl-2 (an inhibitor of apoptosis), and increase
Bax and cleaved-PARP1 (inducers of apoptosis) in a dose-dependent manner. Galangin is also
reported to concurrently induce necrotic pyroptosis, along with apoptosis . . .
The methanolic extract of Calotropis Gigantea induces cancer cell apoptosis
Piperlongumine an inexpensive natural plant medicine used for a thousand years, can inhibit
STAT3 and Akt signaling with decrease in anti-apoptotic Bcl-2 protein expression, and can
epigenetically inhibit histone-modifying enzymes including histone deacetylases HDAC1-4
and HDAC6 and may reduce macrophage activity. Pancreatic and other dangerous tumors are
protected from apoptosis/ferroptosis by overexpression of antioxidant enzymes including
thioredoxin reductase 1 (TrxR1) and peroxiredoxin4 (PRDX4). Piperlongumine, blocks TrxR1
and PRDX4, to enable pancreatic, glioma, liver and other cancer apoptosis. Very inexpensive
combinations of piperlongumine with DCA, metformin, curcumin and/or other cancer
apoptosis enhancers, can safely and synergistically promote tumor apoptosis for brief ctDNA
and other biomarker liquid biopsy enhancement as described herein.
Avermectins including ivermectin an inexpensive ‘off-patent’ dihydro derivative of an
avermectin, is one of the safest and most important drugs known with a long history of use,
after its Nobel Prize winning discovery. Ivermectin can induce PAK1-mediated autophagy,
caspase-dependent apoptosis and immunogenic death in cancer cells via WNT-T cell factor
(TCF), Hippo and Akt/mTOR pathways. Ivermectin can also inhibit cancer stem-like cells.
Hydroxychloroquine and chloroquine, relatively safe, inexpensive ‘off-patent’ drugs widely
used throughout the world, may reduce autophagy by lysosomal inhibition to stimulate
apoptosis in a variety of cancer types which employ autophagy to survive.
Albiziabioside A Conjugated with Dichloroacetate, AlbA-DCA, which can induce intracellular
ROS, alleviate accumulation of lactic acid, selectively kill cancer cells and induce apoptosis
and ferroptosis.

Many apoptosis, ferroptosis and/or necroptosis based medications can have undesirable side effects in cancer-therapeutic dosage regimens, but may be more safely used in brief, non-therapeutic single-dosage or very short-term (one day or less) screening dosages, particularly in relatively small dosage or in synergistic combination with safer agents such as many natural plant products, and/or dichloroacetate and its derivatives. In addition, small amounts of anti-cancer agents such as 5-fluorouracil (5-FU), propranolol and/or other agents can be synergistic in mixtures, such as with dichloroacetate at low, relatively safe dosages. Dichloroacetate, “DCA”, is a prototypical apoptosis inducer for human cancer cells. DCA is rapidly absorbed, widely distributes in vivo, and readily crosses the blood-brain barrier, and rapidly (e.g., within 30 minutes) stimulates PDC activity in a screening patient upon its oral or parenteral administration. Multiple short-term doses of DCA can provide a more sustained increase in PDC activity, while the cancer cells are depleting their glucose stores over 1-3 days as p53 enhancement increases apoptosis. Oral single dose, or short-term daily oral or parenteral doses of DCA may, for example, range from about 5 to about 50 mg/kg, with higher dosages used for one-time or 1 day administration, and lower dosage being administered over 2 or 3 days, respectively.

In this regard, FIG. 1 is a schematic graph of circulating tumor DNA (ctDNA) concentration (and corresponding schematic human cancer tumor size/mass) in the blood of an illustrative cancer screening patient, versus time on a long timeline (perhaps a year or more), as compared to a ctDNA detection limit in the patient's blood for conventional ctDNA test capabilities.

As schematically illustrated in FIG. 1, a mass of cancer cells or solid tumor in a screening patient can be initially have been formed in the past at a time T₀ when it is very small—one or a few cells. The ctDNA concentration in the screening patient's blood from the undiagnosed tumor is shown schematically by line 102, as zero concentration at time T₀, and increasing slowly in time with the increase in size or mass of the tumor. The tumor can grow undetected for a long time, while it is most vulnerable to excision or other treatment, and while relatively few of the cancer cells die naturally to emit only small, relatively undetectable amounts of ctDNA in the patient's bloodstream. The conventional ctDNA detection limit is shown schematically as line 104. The ctDNA concentration 102 in the bloodstream of a person with a growing, undiagnosed cancer cannot be readily detected by conventional ctDNA detection methods until the tumor grows to a size large enough at time Ti to produce enough cancer cells which die and slough off sufficient ctDNA for detection. In the meantime, the cancer has grown, perhaps invaded other tissues or organs, and perhaps undergone further mutation or metastasized to additional distant locations.

In accordance with various embodiments of the present methods, the screening patient is administered a very short-term drug means for enhancing cancer cell death by apoptosis and/or necrotosis, to briefly but significantly increase the amount of ctDNA in the screening patient's bloodstream or other body fluids. Because the purpose of the short-term drug means administration is to just briefly enhance the amount of ctDNA, rather than carry out an effective long-term course of anti-cancer treatment, relatively safe pharmaceutical compounds and mixtures can be used which can briefly enhance broad or generic cancer cell death. In this way, the amount of ctDNA can be briefly increased for better detection by a single dose or very short-term multiple doses to accelerate the death of early, as-yet-undiagnosed cancer cells, to “shed” more ctDNA genetic material to enhance detection in a body fluid sample taken at a subsequent time, preferably when the ctDNA concentration has peaked for ctDNA detection after screening dose administration.

As described herein, there are a large number of drugs which can promote apoptosis in various cancer cells. Importantly, there is a large range of natural compounds, including natural compounds of many long-common human foodstuffs, spices, traditional medicines and dietary supplements which can promote apoptosis and/or reactivate dysfunctional cancer cell apoptosis and ferroptosis. Other inexpensive, long-used, ‘off-patent’ drugs with well-known safety profiles such as ivermectin and dichloroacetate are also capable of selectively stimulating or re-enabling cancer cell apoptosis/ferroptosis. In order to maximize the ctDNA released during the enhanced liquid biopsy time period for detection, asymptomatic cancer screening patients should preferably avoid proapoptotic medication, foodstuffs, spices, and supplements for a period of time before the administration of the proapoptotic reactivation means for briefly enhancing cancer cell apoptosis for liquid biopsy. In this way, early-stage cancer cells or dangerously pre-cancer cells which might release ctDNA upon foodstuff or medication enhancement prior to the pre-screening test time period, can release their ctDNA via apoptosis into the bloodstream upon the timed administration of the screening test apoptosis-enhancing means, to increase the sensitivity for early detection, and to enhance the diagnostic quantitation of larger but as-yet undiagnosed tumors. Preferably, the asymptomatic screening patients should best avoid proaptotic drugs, foods or other compounds (many of which are identified herein) for at least 1 day, and more preferably for at least 2 days before the cancer screen testing, in order to have cancer cell (if present) apoptosis rate at a standard pre-testing minimum common for standardized patient testing. Preferably, cancer screening patients should fast except for water, for at least 12 hours before administration of apoptosis-reactivation means, prior to liquid biopsy sampling. While normal cells tolerate short periods of reduced nutrient availability, rapidly proliferating malignant cells have increased transport mechanisms and needs which can facilitate absorption of the apoptosis-enhancing means. It may also be desirable to “starve” cancer cells prior to liquid biopsy. Nutrient inhibition means for reducing cancer cell nutrient intake are well known

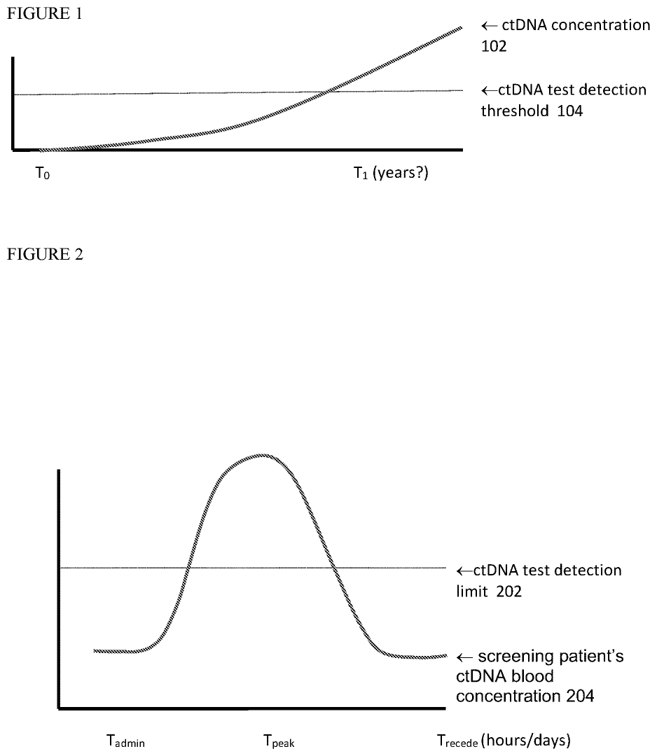

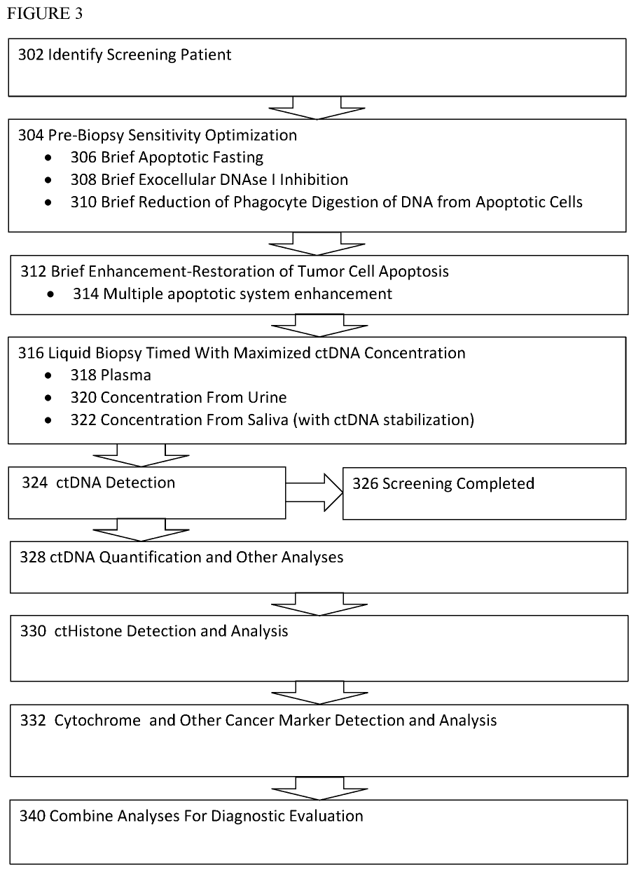

In this regard, FIG. 2 is a schematic graph of circulating tumor DNA (ctDNA) concentration 204 in the blood of a human cancer screening patient having a malignant tumor which is too small to produce adequate ctDNA concentration circulating in the patient's blood to be reliably detected by a conventional ctDNA liquid biopsy test. The screening patient preferably has avoided ingesting proapoptotic foods or medications for at least 1 day (and preferably at least 2 days) and has fasted at least 12 hours (except for water). Then, upon single-dose or short-term administration at time T_{admin} of a cancer apoptosis-inducer, the ctDNA concentration briefly surges to a level above its previous concentration prior to apoptosis-induction, reaching a peak at time Tₚₑₐₖ, which can be detected by ctDNA liquid biopsy test within the time period of ctDNA concentration surge, after which the ctDNA concentration may recede to pre-administration level at time T_{recede} below the ctDNA test sensitivity in the absence of further drug administration. Circulating tumour DNA (ctDNA), which comprises fragmented genomic DNA shed from tumour tissue and circulating freely in the bloodstream (and to a less concentrated extent in urine, saliva and other body fluids), is a current status biomarker for cancer diagnosis. Apoptosis of tumor cells can be induced and ctDNA enhanced within hours or days by DCA and other apoptosis/necroptosis inducing means. Similarly, ctDNA and other circulating nucleic acids can be continuously fragmented and broken down in vivo or eliminated in urine. Upon administration of a bolus or short-term “pulse” of tumor apoptosis/necroptosis inducing means to a cancer-asymptotic screening patient who has an undetected tumor, some of the screening patient's as-yet-undiscovered tumor cells are relatively quickly induced to undergo apoptosis/necrosis, thereby relatively abruptly shedding additional ctDNA and dynamically increasing the concentration of ctDNA in the screening patient's bloodstream (and other body fluids). Because the cfDNA and any of its component ctDNA, if present, is normally biodegraded in vivo, its presence and concentration from an induced enhancement will similarly decrease over a relatively short time period. Subsequently to the short-term apoptosis/necroptosis means administration, the normal body nucleic acid degradation processes reduce the short-term-enhanced ctDNA content of the screening patient's blood (and other fluids), reaching an approximate equilibrium with the previous relative status quo before the administration of the short-term apoptosis/necroptosis drug means, perhaps even reducing below the previous status quo concentration for a period of time.

A cancer screening patient who has a tumor which is advanced enough to produce sufficient ctDNA in the patient's bloodstream for detection without apoptotic/ferroptotic enhancement of ctDNA concentration, will also experience a significant ctDNA bloodstream concentration increase, improving the reliability of the screening test finding of ctDNA and reducing the possibility of a false positive test result. By taking a blood sample at the time of first administration of the apoptosis enhancing agent means, the increase in ctDNA can be quantified, also increasing the reliability of testing.

Reliable detection of ctDNA can be significantly or at least partially diagnostic for the presence of cancer in an asymptotic screening patient's body. Further, identification of the specific tumour-associated mutations found in the detected ctDNA can permit characterization of the type of cancer tissue or organ from which a specific ctDNA has been shed. Degraded nucleic acid components from tumor DNA, identified in circulating free Nucleic acid components of asymptomatic, cancer-undiagnosed screening patient body fluids (cfNA) can also be diagnostic for previously undiagnosed, early-stage cancer. Techniques for isolating cfDNA and ctDNA, and detecting, quantifying, and identifying or otherwise analyzing ctDNA are well developed and include BEAMing, tagged-amplicon deep sequencing, digital PCR, and whole genome sequencing (WGS). A variety of commercial kits with processing instructions are available to detect and capture cfDNA for ctDNA, and/or cfNA analysis. The polymer mediated enrichment PME kit, (Analytik Jena AG, Jena, Germany); the cfPure™ cell-free DNA purification kit (Amsbio, Abingdon, England) using silica-coated paramagnetic particles with a buffer optimized for recovery of 100-500 bp DNA fragments are well-known, as are the E.Z.N.A. Blood DNA Kit (OMEGA Bio-tek, Inc., Norcross, GA, USA); MagCore HF16 Automated DNA/RNA Purification System, MagCore Genomic DNA Whole Blood Kits (RBC Bioscience corp., Taiwan); Polymer-mediated Extraction Kit for cfDNA; QiaAMP cell-free DNA kit, e.g., QiaAmp, to enrich cfDNA from plasma, and ddPCR to measure the resistance mutations (QIAGEN N.V, Hilden, Germany); Maxwell RSC ccfDNA Plasma Kit (RSC) used with Maxwell® RSC Instruments (Promega corporation, Madison. Wisconsin); EpiQuick Circulating Cell-Free DNA Isolation Kit (EQ Epigentek Group Inc., Farmingdale, NY; NEXTprep-Mag cfDNA Isolation Kits (PerkinElmer, Waltham, Massachusetts); QIAamp circulating nucleic acids kit (Qiagen, Germany); Plasma/serum cell free circulating DNA Purification mini kit (Norgen Biotek, Canada), QIAamp minElute ccfDNA mini kit (Qiagen); Maxwell RSC ccfDNA plasma kit (Promega, USA); MagMAX cell-free DNA isolation kit (Applied Biosystems, USA); and NextPrep-Mag cfDNA isolation kit (Bio Scientific, USA) are commercial cfDNA and component ctDNA detection systems. Digital droplet PCR can also be used to quantify the total cfDNA concentration, as well as a mutated ctDNA fraction (if present) such as a KRAS-mutated fraction, and electro-osmotic, microfluidic and lab-on-a-chip technologies can automate ctDNA and/or cfNA detection, as well as quantification and characterization.

Typical conventional cfDNA (and ctDNA if present) detection methods may utilize standard collection of blood samples from patients by venipuncture, centrifugation of the blood samples to remove blood cells and/or other components, and extraction of cfDNA from the plasma. A variety of procedures are conventionally used for concentration, isolation and/or extraction of cfDNA (and ctDNA if present) and/or cfNA from the plasma or other body fluid, which may or may not comprise detection and identification of ctDNA and/or cfNA. Identification of specific DNA sequences in ctDNA can typically be carried out by methods such as sequence specific detection (PCR based) and general genomic analysis of all cfDNA present in the blood (DNA Sequencing including massively parallel sequencing) for ctDNA present in low concentrations in the plasma for example by PCR amplicon sequencing and/or hybrid capture sequencing.

A wide variety of thermally responsive polymers phase separate from their aqueous solutions upon heating. A few polymers exhibit phase separation upon cooling. Such polymers may be modified, tuned and adjusted in their solution and phase separation properties by a wide variety of conventional technologies including copolymerization, end-group design, molecular weight control, crosslinking, grafting, pH adjustment, use of surfactants and electrolytes, effects of co-dispersion components, molecular weight control, and composition blending with other polymers. Examples of thermally responsive amphiphilic polymers include Polyvinyl methyl ether, N-substituted poly[(meth)acrylamide]s, poly(N-vinylamide)s, poly(oxazoline)s, 2-(dimethylamino)ethyl methacrylates, poly(ether)s, polymers based on amphiphilic balance, elastin-like synthetic polymers, and their copolymers, block polymers, blends and grafts. The precipitation of thermosensitive polymers above their lower critical solution temperature (LCST) by change from hydrophilic to hydrophobic character causes water expulsion and aggregation, which can be enhanced by facilitating interpolymer chain association, enhancing intermolecular contacts (e.g., by increasing polymer concentration, enhancing the exterior polymer interface more adhesive or attractive to other polymer clusters. For example, PVME has a glass transition temperature well below ambient temperatures, so can be precipitated from water solution by warming above its LCST to form a rubbery precipitate. PNIPA has a significantly higher glass transition temperature, so upon precipitation from water solution by warming above its LCST can form a harder precipitate. Amphiphilic polymers can be grown from, or grafted to magnetic or ceramic/clay/silica/alumina particles to produce components which are stable in water suspension below the LCST, but can coalesce and aggregate when appropriately heated above the LCST of the amphiphilic polymer(s) to facilitate centrifugal and/or electrostatic collection and/or separation. Copolymer moieties which assist intrapolymer attachment can increase thermosensitive polymer water expulsion and mass coagulation. For example, a small amount of pendant medium-chain alkyl group co-polymer component (e.g., 2-16 carbon n-alkyl vinyl ether) copolymerized at random or endgroup positions in the water-soluble thermosensitive polymer chain can facilitate formation of an extended network at appropriate concentration (e.g., above about 1% by weight of the water component) which enhances coagulation of the polymer above its LCST and expulsion of water when the aqueous build dispersion is heated above its LCST. For example, PVME or PNIPA polymers (together with solid particles coalesced therewith) with pendant DNA groups selective toward cfDNA (and ctDNA if present) can be effective in concentration enhancement of the desired DNA analytes. For example, an additive thermoresponsive aqueous gold nanoparticle ctDNA “methylscape” analytical component can be provided which is thermosensitive to precipitation from aqueous solution/suspension while attracting ctDNA. PVME And PNIPA are water-soluble at low blood-and-plasma preservation temperatures and low red-blood-cell centrifugation speeds and temperatures, but precipitate above their LCSTs (e.g., ˜33° C.) at, say, 37° C. human body temperatures from plasma with attraction of cfDNA/ctDNA for efficient centrifugation collection.

The amount of cancer marker enhancement in body fluid over time can also be useful for diagnosis. A necroptosis and/or apoptosis-inducing drug need not be a “cure” or “clinically effective” for treatment of specific type of cancer, but should preferably be reasonably safe in short-term dosage used. Guided or cell-seeking agents such as drug-loaded nanoparticles selective for specific cancers could also be used at low one-dose, non-effective-treatment amounts, to briefly and selectively enhance the amount of ctDNA for detection at a timed later interval.

Cytochrome c is a relatively small, approximately 14 kDa hemoprotein located at the outer surfaces of inner mitochondrial membranes in functioning cells. In the terminal portions of one type of apoptotic pathway, Cytochrome c can be released into the cell cytosol following initiation of apoptosis, to form an apoptosome with dATP, apoptotic protease activating factor-1 Apaf-1 and procaspase-9, to release caspase-9, which in turn initiates cell proteolysis by executioner caspases 3, 6, and 7. The apoptosis-released cytochrome c can enter the bloodstream of heart attack, trauma, cancer and other ill patients relatively rapidly (e.g., within 1 day) but can also be rapidly eliminated in urine through the kidneys. Cytochrome c is soluble in human sera, so is readily separated from blood cells and other suspended solid components of blood samples by centrifugation and other techniques. The cytochrome c concentration in sera of normal, healthy patients is less than 25 ng/ml of blood sample, while the sera concentration of cancer patients undergoing chemotherapeutic killing of cancer cells can be less than 25 ng/ml but can also range higher (likely for patients with high tumor cell mass which is difficult to successfully treat), so cytochrome blood concentration measurement alone does not appear to be a reliable method for general early cancer detection screening for asymptomatic patients not already diagnosed with cancer. However, measuring increased levels of circulating cytochrome c in body fluids (e.g., blood or urine) upon short-term induction of apoptosis in cancer cells in asymptomatic undiagnosed screening patients, could provide some diagnostic information about the presence of tumor cells which can be combined with ctDNA and/or mutated RNA and mutated protein cancer marker detection data in accordance with conventional methods, to increase the reliability of such screening testing.

Cancer-associated fibroblasts which can contribute a large amount of a tumor's mass, typically highly overexpress fibroblast activation protein (FAP), a serine protease. Accordingly, detection and preferably quantitation of FAP can be important for cancer screening assay, particularly when combined with ctDNA and/or Cytochrome C assay patient screening test information. A variety of conventional test procedures are available to detect and assay FAP content of patient serum and plasma.

Various aspects of the present disclosure are directed to method for detecting cancer in a human patient, which may be an asymptomatic patient without previous diagnosis of cancer, comprising the steps of administering an effective dose to the patient of a drug means for inducing apoptosis and/or ferroptosis in human cancer cells having disabled programmed cell death functions, obtaining a sample of a fluid from the patient within 7 days, preferably within 4 days, of the first administration of the apoptosis and/or ferroptosis inducing drug means, and analyzing the sample fluid from the patient for the presence of ctDNA, cancer marker RNA, cytochrome c, FAP, ctHistone and/or cancer marker proteins.

Preferably the sampled patient fluid is the cancer-screening patient's blood, and the blood is sampled for ctDNA testing at the approximate time of peak ctDNA concentration in the patient's blood induced by the apoptosis and/or necroptosis inducing drug means. The peak enhancement time(s) for ctDNA increase can be determined for various drugs and drug combinations by multiple tests for the ctDNA of treated screening patients over time.

The drug means for inducing apoptosis should best restore, reactivate, accelerate or otherwise induce apoptosis in a wide range of different human cancer cell types. Preferably the human cancer cell apoptosis-inducing drug means should at least double the apoptosis rate of human cancer cells, and more preferably at least increase the apoptosis rate by a factor of at least 10 over the apoptosis rate for cancer cells or tumor within the screening patient's body in the absence of (e.g., immediately prior to) administration of a cancer cell apoptosis inducer. In this regard, for example, if the apoptosis or other death rate of a cancer tumor in a screening patient provides approximately 0.5 ctDNA molecule (which may be undetectable) in a standard 10 ml blood sample immediately prior to initial administration of the cancer cell apoptosis enhancement means, at a peak time within 96 hours after such initial administration, the concentration of ctDNA will most preferably increase at this peak concentration time to about 5 ctDNA molecules per 10 ml of the patient's blood sample. The ctDNA testing may be carried out in accordance with current test technology, and improved ctDNA testing which may be developed in the future.

For general screening of asymptomatic patients who have not previously been diagnosed with cancer, it is preferable to employ an apoptosis-inducing drug means which is relatively safe and has few if any side effects other than facilitating cancer cell apoptosis and/or necroptosis. The cancer apoptosis-inducing drug means can desirably be a mixture of drug compounds, preferably a synergistic mixture which induces or enhances apoptosis in human cancer cells to a greater extent than the sum of the individual components of the mixture. There are multiple apoptosis pathways which are typically compromised in malignant cells, only one of which need be successfully restored to apoptotic functionality, but a number of which may be sufficiently damaged that they are not readily restored or reactivated. Preferably, the cancer cell apoptosis reactivation means is a mixture of at least 3 compounds which each can enhance or restore a different apoptotic pathway, in order to effect apoptosis increase in a broad range of types of cancer cells.

Because the apoptotic drug administration is employed to induce a short-time increase in cancer cell death with concomitant increase in ctDNA in the patient body fluid(s) rather than a long-term effective therapeutic dosage in a cancer treatment window, the dosage level may be tailored to minimize side effects and maximize safety. Synergistic mixtures of different cancer cell apoptosis-inducing agents can reduce the dosage levels of the short-term screening test administration, to facilitate patient safety and reduce side effects. For example, salinomycin is a well-known antibiotic monocarboxylic polyether ionophore with a variety of anticancer mechanisms, without severe side effects at effective dosage levels, which can have a synergistic cancer-cell cytotoxic effect when co-administered with an inhibitor of pyruvate dehydrogenase kinase such as dichloroacetate. A wide variety of natural plant compounds are reported to enhance P53 protein expression, induce apoptosis, reduce expression of proteins P27, P21, and inhibit the PI3K/Akt pathway. Reforming energy metabolism with auraptene, a natural plant citrus extract, known as a mitochondrial inhibitor, can also suppress mitochondrial cancer cell respiration. Co-administration of auraptene with dichloroacetate can have synergistic apoptosis cytotoxic effects on human cancer cells in vivo. Further in this regard, curcumin (including more absorbable and/or physiologically persistent curcumin derivatives) can have synergistic anti-cancer cell effects. Dichloroacetate combined with curcumin can augment inhibition of cancer cell survival, and enhance apoptotic induction accompanied by mitochondria-dependent apoptotic signaling activation. Green tea phytopolyphenols such as (−)-epigallocatechin-3-gallate (EGCG) are reported to induce of cell apoptosis. Garlic and its active compounds including diallyl sulphide, diallyl trisulfide, ajoene and allicin are similarly reported to induce apoptosis.

Catechins, curcumin, genistein, quercetin, berberine and resveratrol, among others, are reported to overcome cancer cell inactivation of tumor suppressor genes. Ginger compounds such as gingerol, paradol, zingiberene and shogaol are reported to upregulate tumour suppressor genes and apoptosis. Sulforaphane (1-isothiocyanato-4-(methyl-sulfinyl)-butane), a relatively safe compound derived from cruciferous vegetables, is reported to induce apoptosis in cancer cells by a variety of mechanisms. Such synergistic apoptosis activation in cancer cells can lower the dichloroacetate dose employed to temporarily increase the concentration of ctDNA in a cancer-screening patient's bloodstream or other bodily fluid, for earlier cancer detection.

Dichloroacetate (DCA) is an unpatented very low cost pyruvate dehydrogenase kinase inhibitor. It can reverse the Warburg effect by switching ATP production back to oxidative phosphorylation, reduce mitochondrial membrane potential (ΨIM), and activate mitochondrial potassium channels, to contribute to the induction of apoptosis in various cancers through the release of cytochrome c (cyt c) and apoptosis inducing factor (AIF). Dichloroacetate is an inexpensive, prototypic xenobiotic inhibitor of PDK, thereby maintaining PDC in its unphosphorylated, catalytically active form. Similarly there is a wide variety of very inexpensive compounds and extracts such as those present in natural food and medical plants that have been used and observed for extended periods of time, which are capable of repairing or inducing apoptosis in cancer cells. Long-used, inexpensive and safety-tested compounds such as aruptane, curcumin and other natural or FDA-approved and safe apoptosis-inducing means which can be synergistically combined with DCA and/or its derivatives are important for enabling very-inexpensive screening to control cost for screening for the cancer-asymptomatic general population, for early cancer detection.

Cytochrome c is a relatively small, approximately 14 kDa hemoprotein located at the outer surfaces of inner mitochondrial membranes in functioning cells. In the terminal portions of one type of apoptotic pathway, Cytochrome c can be released into the cell cytosol following initiation of apoptosis, to form an apoptosome with dATP, apoptotic protease activating factor-1 Apaf-1 and procaspase-9, to release caspase-9, which in turn initiates cell proteolysis by executioner caspases 3, 6, and 7. The apoptosis-released cytochrome c can enter the bloodstream of heart attack, trauma, cancer and other ill patients relatively rapidly (e.g., within 1 day) but can also be rapidly eliminated in urine through the kidneys.

Cytochrome c is soluble in human sera, so is readily separated from blood cells and other suspended components of blood samples by centrifugation and other techniques. The cytochrome c concentration in sera of normal, healthy patients is less than 25 ng/ml of blood sample, while the sera concentration of cancer patients undergoing chemotherapeutic killing of cancer cells can be less than 25 ng/ml but can also range higher (likely for patients with high tumor cell mass which is difficult to successfully treat), so cytochrome c blood concentration measurement alone does not appear to be a very reliable method for general early cancer detection screening for asymptomatic patients not already diagnosed with cancer. However, measuring increased levels of circulating cytochrome c in body fluids (e.g., blood or urine) upon short-term induction of apoptosis in cancer cells in asymptomatic undiagnosed screening patients, could provide diagnostic information about the presence of tumor cells which can be combined with ctDNA detection data to increase the reliability of such screening testing.

The stimulation and enablement of impaired or dysfunctional programmed cell death apoptosos/ferroptosis pathways in human tumors facilitates earlier detection of cancer biomarkers by liquid biopsy by increasing the amount of cancer biomarker(s) such as ctDNA to a detectable (or assayable) level which could otherwise be below the detection limit of the detection (or assay) utilized for the liquid biopsy sample. There are many well-known detection methods, kits and procedures, with new methods, kits and procedures being developed, which are limited by the unstimulated small amounts of circulating ctDNA, ctHistones and other cancer biomarkers normally present from small early-stage tumors. Examples of doses of means for inducing short-term apoptosis for increasing ctDNA and other cancer biomarkers in blood of cancer screening patients for blood sample taken for ctDNA testing within 4 days of first dosage administration, preferably within 2 days of first dosage administration for simplicity and cost reduction:
25 mg/kg DCA orally on day 1, blood sample for ctDNA and/or cfNA testing and determination of the presence of ctDNA may be taken on day 2, 24 hours after the DCA administration. Alternatively, the blood sample(s) may be taken from the patient at an optimal ctDNA and/or cfNA sampling time for the peak concentration enhancement of ctDNA and/or cfNA in patient's blood (or other body fluid) may be determined by selecting a cohort of 1,000 cancer-asymptomatic screening patients, say those over 50 years old, with genetic relatives who have had cancer, or otherwise by genetic or environmental factors are at enhanced cancer risk, administering the cancer-cell-apoptosis inducing means, and sampling their respective body fluid (e.g., blood, every hour for other useful est interval for at least 4 days) for the presence of ctDNA (and/or cancer-characteristic cfNA), and determining the peak concentration time for the desired analyte(s). Those screening patients who are cancer free should have no detected cfDNA. Those who have an early stage, undiagnosed cancer may have no cfDNA detected in early blood samples (e.g., taken at the time of dose administration and one hour after dose administration) but will later show the presence of enhanced ctDNA concentration (e.g., starting and peaking several hours or even days after the initial dose administration time) to provide a curve 204 like that of FIG. 2. Other cancer-asymptomatic screening patients with larger or later-stage tumors may show a detectable amount of ctDNA by conventional ctDNA test at the time of initial dose administration, with subsequently increasing amounts of ctDNA until a peak is reached, then a declining concentration of ctDNA, following a curve 204 like that of FIG. 2 but starting and ending above the detection limit 202 for the ctDNA Test utilized.
The overall best detection sampling peak can be selected from this type of cohort test data.
Same as the immediately above protocol, plus a 50,000 IU dose of vitamin D3 administered orally on day one with the 25 mg/kg DCA.
25 mg/kg DCA orally on day 1, 7 mM DCA orally on day 2, blood sample for ctDNA testing taken on day 3, 48 hours after first administration. Peak single-sample blood sampling time for low-cost screening can be determined as above or by similar protocol.
20 mg/kg DCA orally on day 1, 10 mM DCA orally on day 2, 5 mM DCA orally on Day 3, blood sample for ctDNA testing taken on day 4, 72 hours after first administration. Peak single-sample blood sampling time for low-cost screening can be determined as above or by similar protocol.
0.25 μM of salinomycin and 20 mg/kg of DCA on day 1, 10 mg/kg DCA on day 2, blood sample for ctDNA testing taken on Day 3, 72 hours after first administration. Peak single-sample blood sampling time for low-cost screening can be determined as above or by similar protocol.
20 mg/kg DCA plus 1 gram curcumin (diferuloylmethane) on day 1, ctDNA blood sample on day 2, 36 hours after first administration. Peak single-sample blood sampling time for low-cost screening can be determined as above or by similar protocol.
25 mg/kg DCA plus 1 gram curcumin day 1, 10 mM DCA plus 1 gram curcumin day 2, ctDNA blood sample day 3, 48 hours after first administration. Peak single-sample blood sampling time for low-cost screening can be determined as above or by similar protocol.
20 grams of Granular micronized Salvia miltiorrhiza powder, prepared and suspended in 200 ml water, together with 25 mg/kg DCA are administered orally to asympromatic cancer screening patients on Day 1. A blood sample is taken and tested for ctDNA 12 hours, and 24 hours after administration. The test may be an inexpensive University of Queensland “methylscape” or other conventional test. Those screening patients who test positive for ctDNA are referred to subsequent examination and treatment as appropriate.
20 grams of Granular micronized Salvia miltiorrhiza powder prepared and suspended in 200 ml water, 50 mg/kg of green tea Epigallocatechin-3-gallate, and 20 mg/kg DCA are administered orally to asymptomatic early cancer detection screening patients to provide a variety of cancer cell apoptosis reactivation mechanisms (p53, p16 . . . ). Blood samples are taken immediately prior to administration, and at 6 hours and 24 hours after administration. The blood samples are tested for ctDNA, mutated RNA cancer markers, mutated protein cancer markers, and cytochrome c.
40 mg propranolol, 50 mg/kg green tea Epigallocatechin-3-gallate, 25 mg/kg DCA and 400 mcg Sulforaphane are administered orally to asymptomatic early detection cancer screening patients to restore or reactivate a variety of cancer cell apoptosis pathways. Blood samples are taken immediately prior to administration administration, and at 6 hours, 12 hours and 24 hours after administration. The blood samples are tested for ctDNA, mutated RNA cancer markers, mutated protein cancer markers, and cytochrome c.
Asymptomatic cancer screening patients instructed to avoid apoptosis-inducing foods, supplements and/or non-prescribed medication for 2 days, and to fast completely (except for water) for 12 hours, are then administered a mixture of epigallocatechin-gallate (800 mg, chloroquine (500 mg chloroquine phosphate−300 mg base) or 300 mg hydroxychloroquine, 20 mg/kg piperlongumine, and 15 mg/kg DCA. Liquid biopsy samples are taken after 6 hours and 12 hours, for assay of ctDNA, mutated RNA cancer markers, mutated protein cancer markers, cytochrome c, and FAP, for evaluation of abnormally elevated levels.
Cancer screening patients are administered Ivermectin at a dosage of 0.50 to 0.75 mg/kg. Liquid biopsy samples are taken immediately prior to Ivermectin administration, at 6 hours, and at 24 hours after administration for assay of ctDNA, ctHistones, mutated RNA cancer markers, mutated protein cancer markers, cytochrome c, and FAP, for evaluation of abnormally elevated levels.
Cancer screening patients are administered Ivermectin at a dosage of 0.50 to 0.75 mg/kg, 20 mg/kg piperlongumine, and 15 mg/kg DCA. Liquid biopsy samples are taken immediately prior to administration, at 6 hours, and at 24 hours after administration for assay of ctDNA, ctHistones, mutated RNA cancer markers, mutated protein cancer markers, cytochrome c, and FAP, for evaluation of abnormally elevated levels, for cancer detection.

The peak ctDNA and/or cancer-defining cfNA concentration times in screening patients may be determined by testing of a specific dose regimen in a cohort of asymptomatic screening patients. For simplicity, cost reduction and standardization, a single body fluid sample (e.g., blood sample) may be taken for testing at a standardized peak concentration time for the cancer marker(s) being assayed, after the time of initiation of the apoptosis restoration means. A sample may also be taken at the time of initial administration of the apoptosis restoration means for minimizing additional cost, so that changes in concentration (amount) of the cancer marker(s) ctDNA, RNA, proteins and/or cytochrome c before and after the administration can be determined. For each of these and other screening tests, an initial screening patient cohort may have blood samples taken periodically to establish the peak ctDNA and/or cfNA concentration time(s) after first apoptosis and/or necroptosis inducer administration to facilitate use of the peak concentration sampling time for body fluid sampling for detection.

The disclosed apoptosis/ferroptosis circulating cancer marker enhancement methods for screening asymptomatic patients for the presence of cancer cell ctDNA may also be applied to the enhancement of cell-free circulating nucleic acids (cfNA) which can be produced by natural in vivo destruction of cfDNA including ctDNA (e.g., by DNAse), RNA, proteins and cytochrome c (as a general measure of apoptosis, not necessarily from cancer cell apoptosis) and detection and analysis by conventional methods such as described in cited references. For example, for each of the blood samples described in the above enhanced apoptosis, asymptomatic patient screening tests, ctDNA detection and analysis may be carried out in accordance with conventional methods, and may be combined with detection and analysis of cytochrome c, cancer marker proteins present in cancer cells, and/or malignant-marker RNA in accordance with conventional practice, for statistically more accurate screening. For example, in accordance with the conventional CancerSEEK™ ctDNA testing. In this regard, a panel of protein and gene markers, and/or RNA markers, protein markers, and/or cytochrome c apoptosis marker may be used to screen for tumors

A 61-amplicon panel, with each amplicon querying an average of 33 base pairs (bp) within one of 16 genes may be used to detect and analyze the enhanced amount (concentration) of ctDNA in the screening patient blood provided by short-term cancer cell apoptosis re-enablement, with published Primer sequences for multiplex PCR assays.

Multiplex-PCR may be applied to label each original template molecule with a unique DNA barcode to reduce massively parallel sequencing error, and multiassays may be used to increase signal to noise analysis ratio in accordance with the conventional CancerSEEK™ processing. Further in accordance with a CancerSEEK™ type ctDNA cancer screen processing, the blood samples may each be analyzed for protein markers for malignancy. In this regard, 8 proteins known to detect at least one of eight cancer types may be reproducibly evaluated through a single immunoassay platform used to assay plasma samples in accordance with the CancerSEEK™ protocol, summarized in Table 2 below.

TABLE 2
Protein biomarkers which can be analyzed,
and those included in CancerSEEK test

| | Included in |
|---|---|
| PROTEIN | CancerSEEK ™ test |
| AFP | No |
| Angiopoietin-2 | No |
| AXL | No |
| CA-125 | Yes |
| CA 15-3 | No |
| CA19-9 | Yes |
| CD44 | No |
| CEA | Yes |
| CYFRA 21-1 | No |
| DKK1 | No |
| Endoglin | No |
| FGF2 | No |
| Follistatin | No |
| Galectin-3 | No |
| G-CSF | No |
| GDF15 | No |
| HE4 | No |
| HGF | Yes |
| IL-6 | No |
| IL-8 | No |
| Kallikrein-6 | No |
| Leptin | No |
| LRG-1 | No |
| Mesothelin | No |
| Midkine | No |
| Myeloperoxidase | Yes |
| NSE | No |
| OPG | No |
| OPN | Yes |
| PAR | No |
| Prolactin | Yes |
| sEGFR | No |
| sFas | No |
| SHBG | No |
| sHER2/sEGFR2/sErbB2 | No |
| sPECAM-1 | No |
| TGFa | No |
| Thrombospondin-2 | No |
| TIMP-1 | Yes |
| TIMP-2 | No |
| Vitronectin | No |

A wide variety of devices, kits, methods and techniques are capable of successfully detecting, interpreting, and monitoring ctDNA and other cancer biomarkers in body fluids, including blood and urine. Precise information about malignancies can be obtained from liquid biopsies by isolating and analyzing circulating tumor DNA, nucleic acids, cytochrome c, FAP, ctHistones, tumor-derived vesicles, proteins, and/or metabolites.

A significant amount of ctDNA from cancerous apoptotic cells (and DNA from non-cancerous apoptotic cells) may be destroyed before entering the bloodstream, by phagocytic systems which digest apoptotic cells and their contents. In accordance with some aspects of the present disclosure, phagocyte activity which degrades DNA from apoptotic cells may be briefly inhibited with appropriate timing to decrease the amount of DNA from apoptotic cells digested by phagocytes, and to thereby increase the amount of tumor cell DNA released into the bloodstream as ctDNA for increased liquid biopsy concentration and sensitivity. Macrophages may be inhibited in a variety of ways to reduce digestion of apoptotic DNA, thereby increasing the amount of DNA released as cfDNA from apoptotic cells. In this regard, CNI-1493, semapimod, is a synthetic guanylhydrazone which inhibits macrophage activation as well as production of several inflammatory cytokines. Macrophage migration inhibitory factor inhibitor 3-[(biphenyl-4-ylcarbonyl) carbamothioyl]amino benzoic acid (Z-590) suppresses macrophage activation. Similarly, macrophage activation and engulfment systems for apoptotic cells may be inhibited. In one major phagocytic system, upon apoptosis phosphatidylserine molecules are transferred from the inner leaflet of the plasma membrane to the extracellular surfaces of apoptotic cells, as markers which induce macrophages to attach and digest them. Macrophage receptors directly or indirectly recognize phosphatidylserine cell surface markers to trigger apoptotic cell engulfment and digestion. Apoptotic cells can also release apoptotic bodies containing their DNA, which bodies can be ingested by adjacent cells including macrophages and dendritic cells for complete digestion by DNaseII in lysosomes, further limiting the amount of cfDNA, and ctDNA from tumor cells if present, which enter the bloodstream. Translocation of phosphatidylserine from the inner leaflet to the outer leaflet of the endothelial membrane is carried out by phospholipid scramblase-1 (PLSCR1). Inhibiting phosphatidylserine expression at apoptotic cell surfaces can accordingly be applied herein in conjunction with optimization of liquid biopsy timing, to reduce the efficiency of phagocyte destruction of DNA released from apoptotic cells, thereby increasing the amount of cfDNA released into the bloodstream, including ctDNA from a patient's apoptotic tumor cells. Inhibition of phosphatidylserine exposure may be carried out by brief administration of effective amounts of inhibitors of PLSCR1 translocation such as dithioerythritol), (2) inhibition of PLSCR1 membrane trafficking (2-bromopalmitate), and inhibition of ion exchange necessary for PLSCR1 function (4,4′-Diisothiocyano-2,2′-stilbenedisulfonic acid). Targeted inhibitors of phosphatidylserine exposure include knockdown of PLSCR1 using RNA interference (RNAi), and Diannexin. Resveratrol is also an inhibitor of expression of PLSCR1, while anthelminthic drug niclosamide may inhibit Anoctamin ANO1 (of the TMEM16 protein family of chloride channels and phospholipid scramblases) to regulate exocytosis.

As well known, and described herein, a very wide variety of methods and analytical kits and devices are well known and available for ctDNA and other cancer biomarker detection, quantification and analyses. Gold oxide/hydroxide components, including nanoparticles, may be formed on a suitable substrate with a TiO2 surface, such as a plasmonic design on an optical fiber, magnetic particles, or transparent substrates, and then reduced by exposure to a reducing agent and/or UV or blue VIS light to form Au particles or surfaces. Mild reducing agents which have little or no effect on ctDNA, such as citric or ascorbic acid, may be used to reduce gold oxide or hydroxide particles or zones to metallic gold which preferentially adsorbs ctDNA. Ultraviolet UVA light (from about 315 to 400 nm) and blue light (from about 400 nm to about 500 nm) have relatively low absorption by, or effect on, DNA (as compared to UVB) which has higher energy and DNA absorption spectrum. In situ gold nanoparticle formation can enhance ctDNA adsorption thereon, for colorimetric and/or electrochemical detection and analysis.

Nanoparticles with both magnetic and optical plasmonic properties, comprised of a magnetic iron-oxide core with an external gold nanoscale coating such as nanoparticles having localized plasmon resonance properties are well-known in preparation and use. The external gold surfaces can be conventionally functionalized by a variety of surface chemistries, if desired, for dispersion and compatibility in biological media to isolate and purify DNA and biological proteins. The optical properties of such nanocomposites are conventionally useful for plasmon-enhanced and other processes for the detection of specific trace analytes in aqueous media.

Single-stranded DNA (ssDNA) including single stranded ctDNA (ss-ctDNA), can be present in liquid biopsy samples, and is also readily detected by colorimetric plasmonic and electrochemical response of gold nanoparticles and electrode surfaces; ss-ctDNA can have some advantages for conventional diagnostic library preparation. Noble metal nanoparticles can be conventionally formed in nanoscale arrays by employing top-down and/or bottom-up liquid-based self-assembly, to provide plasmonic and other optical-detection devices by methods such as directly linking the nanoparticles through linker molecules, and/or decorating a preformed template with the nanoparticles.

Extracorporeal ctDNA may also be collected and preserved against degradation by adsorption or other attachment to a preservation substrate such as a hydroxyapatite surface, Montmorillonite clay, and the like. The affinity of DNA binding to hydroxyapatite stabilizes the affinitively bound DNA, making it resistant to degradation by nucleases, and facilitating cfDNA accumulation (including ctDNA if present). Accumulated cfDNA including ctDNA if present, may be released from the hydroxyapatite using EDTA and/or other conventional DNA harvest or release processing methods. In this regard, it may be advantageous in some circumstances to screen patients without taking invasive blood and plasma samples. However, while the amount of cfDNA in blood and plasma samples is small, it is much less in screening patient fluids such as saliva and urine. Accordingly, brief enhancement of tumor apoptosis/ferroptosis to significantly increase the concentration of ctDNA and other cancer biomarkers is a significant and important enhancement for cancer detection methods which utilize extracorporeal patient fluids such as urine and saliva. Further in accordance with the present disclosure, the briefly enhanced amounts of cfDNA in saliva or urine may be further concentrated from patient fluids such as saliva and/or urine by collection from large volumes of fluid over time while preserving the collected cfDNA. For example, polyethyleneimine may be combined with phosphate inorganic compounds such as hydroxyapatite or iron phosphate to provide a DNA absorbent and DNA preservation means for collecting and preserving ctDNA from saliva or urine. A crosslinked coating for porous cellulose cloth mouthguards which can be retained in the mouth for extended time periods (e.g., overnight) may be employed to collect cfDNA for detection of the presence of ctDNA, as well as subsequent ctDNA quantification and/or analysis.

In order to increase the concentration and amount of ctDNA, as well as ctHistones for patient cancer screening or other testing, oral cavity collection means may be positioned in the patient's oral cavity over a period of time for collecting DNA including ctDNA, and/or Histones including ctHistones, which may be present in the patient's saliva. For example, a conventional mouthguard type device may have removable DNA and Histone absorbing inserts, such that the inserts are in contact with saliva in the patient's mouth and collect DNA and Histones from the saliva. The oral cavity DNA and Histone collection means may be retained in the patient's mouth for an extended period of time, for example, conveniently overnight or longer, for extended collection from the significant quantity of saliva continuously generated by the patient.

Histones are well conserved basic proteins which associate to form an octameric core around which cell DNA is wound to form a nucleosome. N-terminal tails of histones extend from the nucleosome and undergo a variety of post-translational modifications including acetylation and methylation. Histone Post Translational modifications are conventionally analyzed and characterized by well-known chemical and biochemical procedures, for example by high-throughput sequencing and DNA-microarray analysis techniques for conventional mapping of the epigenome, and detection of human cancer cells.

The amount and/or concentration of cfDNA is relatively low, and the amount/concentration of ctDNA if present is much lower. In the case of patients with small, early tumors, the detection of the smallest amounts ctDNA which can be reliably detected determine the earliest stage at which the presence of the tumor can be recognized by liquid biopsy detection and/or analysis. Accordingly, it is conventional to collect, concentrate and analyze ctDNA. In accordance with some aspects of the present disclosure, the dilute ctDNA component(s) of the liquid biopsy sample fluid of a patient may be separated and concentrated for detection. One method for concentration and detection is contacting the liquid biopsy sample, such as plasma, urine or saliva, with a ctDNA coordinating metal or conductor such as gold, silver or graphene to selectively collect, adsorb and separate ctDNA and/or other cancer biomarkers such as ctHistones from the patient fluid, and analyze collected histones for methylation and acetylation patterns. Circulating cfDNA (including ctDNA if present) can be bound to histone from the cell(s) of origin (e.g., H3K27me2) which may be involved in both externalization and stability of plasma DNA, and other histones. Relatively short fragments of cfDNA (including ctDNA which may be present) can be partially stabilized against destruction in the bloodstream by association with histones and/or lipoproteins. Like ctDNA, ctHistones are typically aberrant, so can be diagnostic for tumor type as described herein. By combining analysis of ctDNA and ctHistone characteristics, more accurate diagnosis of tumor type and location may be carried out.

Circulating cell-free DNA (cfDNA) comprises small DNA fragments derived from normal cells, and when present, from tumor cells, that are released into the screening patient's body fluids such as blood, urine, cerebrospinal fluid, and saliva. Methylation profiling and/or molecular/structural assay of ctDNA can identify tissue-specific markers in ctDNA. Methods for analyzing ctDNA methylation sites, patterns, methylome and/or tissues or organs of origin are well known. Methylated DNA immunoprecipitation and high-throughput sequencing (cfMeDIP-seq) is a known, sensitive, low-input, cost-efficient and bisulfite-free approach to profiling DNA methylomes of plasma cfDNA. Conventional droplet digital PCR (ddPCR) assays can provide sensitive detection and quantification of target ctDNA molecules using appropriately labelled probes. Electrochemical biosensing of ctDNA and surrogate markers is also a well-known method for assessing tumor burden, heterogeneity of cancer genetic/epigenetic patient assay, and mapping detected ctDNA characteristic features, including tumor-specific mutations, chromosomal aberrations, microsatellite alterations, and epigenetic changes. Specific assay of a screening patient's ctDNA after separation by screening in accordance with the present disclosure can characterize the potential tumor types and location, and aid in the diagnosis, and selection of treatment for the tumor-cells-of origin for the screening-detected ctDNA. Gold-silica nanoparticles functionalized with selective oligonucleotides are conventionally used to detect hypermethylated ctDNA in plasma samples, based on differences in size distribution of nanoparticles hybridized for ctDNA compared with normal DNA functionalization, evaluated by dynamic light scattering. Conventional analysis of ctDNA permits characterization of the mutational status of tumors. Specific patterns of hypermethylation are characteristic of ctDNA. Specific abnormal hypomethylation of DNA repetitive sequences is also a known characteristic of cancer cells.

The very low concentration of ctDNA in saliva can be improved by concentration and long term collection. DNA enrichment from body fluids can comprise DNA extraction such as by means of magnetic beads and polymer-mediated enrichment of DNA. Also in this regard, an oral denture which is designed for retention in the screening patient's oral cavity for at least 3 hours, and preferably at least 8 hours, may be employed to collect and stabilize ctDNA. Overnight retention is a convenient time for the screening patient. The oral ctDNA collection means may comprise a selective DNA means for selective collecting DNA from saliva, which is preferably means for selectively collecting ctDNA such as Au nanoparticles or other Au surfaces. It is noted that DNAses and other DNA-active enzymes are contained in body fluids which are capable of hydrolyzing or otherwise depolymerizing DNA (including ctDNA). Accordingly, the oral cavity collection means may further comprise means for protecting DNA from degradation. The DNA collection device may be in the form of a dental mouthguard molded to the upper and/or lower palate of the screening patient. After a predetermined collection time in the patient's oral cavity, the saliva collection means is removed, and the collected DNA is extracted. The method of extraction may be tailored to the specific DNA collection system. For example, ctDNA collected on suitable gold nanoparticles may be extracted by separating the gold nanoparticles with adsorbed ctDNA from the mouthguard body.

The direct administration of aqueous H₂O₂ into solid tumours can cause tumour cell death, with two distinct advantages over conventional chemotherapeutic agents: to produce minimal short- and long-term side-effects and is relatively cheap and cost effective. The direct administration of hydrogen peroxide to the tumor(s) is conventionally carried out with radiation therapy The direct administration of hydrogen peroxide can be made more synergistically effective by co-administering a thioredoxin reductase 1 (TrxR1) and/or peroxiredoxin (PRDX) blocker such as Piperlongumine (which blocks TrxR1 and PRDX4,) to enable and stimulate apoptosis induced by ROS. The thioredoxin reductase and/or peroxiredoxin blocker can be administered orally, and/or directly to the tumor site, preferably before the direct administration of the hydrogen peroxide.

Having described the disclosure in detail, it will be apparent that modifications and variations are possible without departing from the scope of the claims.

While various aspects of the present disclosure have been described with respect to particular embodiments, it will be appreciated that modifications, applications, adaptations and improvements may be made based on the present disclosure, and are intended to be within the scope of the accompanying claims.

### EXAMPLES

The following non-limiting examples are provided to further illustrate the present disclosure.

### Example 1

As a prospective example, cancer patients with breast tumors ≥3 cm (surgically or medically inoperable) receive conventional published treatment with intratumoral H₂O₂ with either 36 Gy external beam radiation treatment (RT) in 6 twice-weekly fractions (n=6) or 49.5 GSy in 18 daily fractions (n=6) to the whole breast±locoregional lymph nodes. H₂O₂ is mixed in 1% sodium hyaluronate gel (final H₂O₂ concentration 0.5%) before administration to slow drug release and minimize local discomfort. The mixture is injected intratumorally under ultrasound guidance twice weekly 1 hour before RT. Prior to the H₂O₂ administration, the thioredoxin and/or peroxiredoxin inhibitor may be administered orally (for whole-body incorporation) and/or injection in suitable aqueous suspension or solution at effective concentration. For example, 40-100 mg/kg piperlongumine whole body oral administration 1 hour before H₂O₂ direct administration, or 150 mg/kg of piperlongumine based on estimated tumor mass directly injected in solution or suspension (including nanoparticle suspensions). 15 mg/kg DCA based on tumor mass may be included with the piperlongumine. The primary endpoint is patient-reported maximum intratumoral pain intensity before and 24 hours postinjection. Secondary endpoints may include grade ≥3 skin toxicity and tumor response by ultrasound. Blood samples may be collected before, during, and at the end of treatment for cell-death, ctDNA and immune marker analysis.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description shall be interpreted as illustrative and not in a limiting sense.

1. A method for detecting early-stage human cancer in an asymptotic human cancer screening patient not previously diagnosed with cancer and who has free circulating bloodstream cfDNA including a natural amount and concentration of circulating tumor DNA (ctDNA) shed by some cancer cells of a malignant tumor if present within the patient, the cancer cells of which have impaired or inactivated apoptosis functionality which impairs their natural programmed cell death, comprising the steps of:
administering short-term safe, effective dosages to the cancer screening patient of multiple apoptosis pathway drug means for selectively reactivating or inducing apoptosis across multiple apoptosis pathways in human cancer cells to release ctDNA into the patient's bloodstream to increase the amount and concentration of ctDNA in the patient's bloodstream and other bodily fluids above the natural amount and concentration of ctDNA, without substantial harm to normal nonmalignant patient cells, wherein said multiple apoptosis pathway drug means comprises at least 3 apoptotic pathway means for reactivating or restoring apoptosis pathways which are downregulated or otherwise functionally deactivated or silenced in malignant human cancer cells, selected from:
p53 means for activating p53-induced apoptosis in cancer cells,
PDK inhibition means for inhibiting pyruvate dehydrogenase kinases to reduce aerobic glycolysis in cancer cells,
mTOR means for inhibiting the pI3K/Akt/mTOR signaling pathway,
Bax-Bcl2 ratio means for increasing the Bax:Blc-2 cancer cell ratio,
caspase expression means for increasing cancer cell caspase expression of caspase-3, caspase-7 or caspase-9,
DNA demethylation means for reducing cancer cell DNA hypermethylation,
tumor suppressor upregulation means for upregulating at least 2 of tumor suppressor genes DLC1, PPARG, ST7, FOXO6, TET2, CYP1B1, SALL4, and DDIT3, together with upregulation of at least two of pro-apoptotic genes RASL11B, RASD1, GNG3, BAD, and BIK,
histone demethylase inhibition means for inhibiting histone demethylase to restore functional cancer cell histone methylation for cancer cell apoptosis initiation,
ferroptosis stimulation means for stimulating ferroptosis induced apoptosis in cancer cells,
lysosomotropic means for increasing the permeability of lysosomes and enhancing apoptosis, and
means for enhancing pro-apoptotic TRAIL death receptor pathway;
discontinuing the administration of said multiple apoptosis pathway drug means for selectively inducing apoptosis in human cancer cells within 3 days of the initial administration;
obtaining a sample of a body fluid from the patient during said increase in the amount and concentration of ctDNA in the patient's bloodstream and other bodily fluids within 7 days of the initial administration of the apoptosis inducing drug means; and
analyzing the sample fluid from the patient for the presence of circulating tumor DNA and/or cancer-characteristic circulating biomarkers.

2. The cancer screening method of claim 1 for detecting early-stage human cancer wherein said multiple apoptosis pathway drug means consists of natural, safe plant compounds.

3. The cancer screening method of claim 1 for detecting early-stage human cancer wherein said multiple apoptosis pathway drug means comprises dichloroacetate, ivermectin, hydroxychloroquine, plant flavonoids, curcumin, piperlongumine, and/or epigallocatechin-3-gallate.

4. The cancer screening method of claim 1 for detecting early-stage human cancer wherein said multiple apoptosis pathway drug means comprises at least 4 apoptotic pathway means for reactivating or restoring apoptosis pathways which are downregulated or otherwise functionally deactivated or silenced in malignant human cancer cells, selected from:
p53 means for activating p53-induced apoptosis in cancer cells,
PDK inhibition means for inhibiting pyruvate dehydrogenase kinases to reduce aerobic glycolysis in cancer cells,
mTOR means for inhibiting the pI3K/Akt/mTOR signaling pathway,
Bax-Bcl2 ratio means for increasing the Bax:Blc-2 cancer cell ratio,
caspase expression means for increasing cancer cell caspase expression of caspase-3, caspase-7 or caspase-9,
DNA demethylation means for reducing cancer cell DNA hypermethylation, tumor suppressor upregulation means for upregulating at least 2 of tumor suppressor genes DLC1, PPARG, ST7, FOXO6, TET2, CYP1B1, SALL4, and DDIT3, together with upregulation of at least two of pro-apoptotic genes RASL11B, RASD1, GNG3, BAD, and BIK,
histone demethylase inhibition means for inhibiting histone demethylase to restore functional cancer cell histone methylation for cancer cell apoptosis initiation,
ferroptosis stimulation means for stimulating ferroptosis induced apoptosis in cancer cells,
lysomotropic means for increasing the permeability of lysosomes and enhancing apoptosis, and
means for enhancing pro-apoptotic TRAIL death receptor pathway.

5. The cancer screening method of claim 1 for detecting early-stage human cancer, further comprising the step of briefly administering means for inhibiting degradation of ctDNA in the patient's bloodstream while the amount and concentration of ctDNA is being increased in the patient's bloodstream by apoptosis of malignant cells induced by said administration of multiple apoptosis pathway drug means, whereby coordinating said brief increase in apoptotic release of ctDNA into the patient's bloodstream while briefly inhibiting degradation of ctDNA in the bloodstream, the concentration and amount of ctDNA is further increased for detection in the screening patient's blood and other body fluids, wherein said sample of a body fluid obtained from said patient.

6. The cancer screening method of claim 6 wherein said means for inhibiting degradation of ctDNA in the patient's bloodstream comprises a DNAse1 inhibitor somatostatin, MG299-fF35 from M. echinospora, N. tabacum cell culture DNAse 1 inhibitor, actinomycin D, nogalamycin, daunomycin, neomycin B and/or paromomycin.

7. The cancer screening method of claim 1 for detecting early-stage human cancer wherein said p53 means for activating p53-induced apoptosis in cancer cells comprises scrophularia atropatana extract, curcumin, sulforaphane, cardanol monoene, S-Adenosyl-1-methionine, and/or berberine.
wherein said PDK inhibition means for inhibiting pyruvate dehydrogenase kinases to reduce aerobic glycolysis in cancer cells comprises dichloroacetate,
wherein said mTOR means for inhibiting the pI3K/Akt/mTOR signaling pathway comprises pathway comprises piperlongumine, myricetin, caffeine with chloroquine, and/or perifosine
wherein said Bax-Bcl2 ratio means for increasing the Bax:Blc-2 cancer cell ratio comprises tanshinone hydrophobic abietane diterpenes, chinese bayberry leaf flavonoids, bilobetin, isoginkgetin, mycoepoxydiene, piperlongumine, and/or cardanol monoene
wherein said caspase expression means for increasing cancer cell caspase expression comprises chinese bayberry leaf flavonoid, myricitrin, quercetin, oregano, galangin, betulin, and/or betulinic acid and its esters,
wherein said DNA demethylation means for reducing cancer cell DNA hypermethylation comprises hinokitiol, S. atropatana extract and/or epigallocatechin-gallate alone or in combination with eugenol-amarogentin,
wherein said tumor suppressor upregulation means for upregulating at least 2 of tumor suppressor genes DLC1, PPARG, ST7, FOXO6, TET2, CYP1B1, SALL4, and DDIT3, together with upregulation of at least two of pro-apoptotic genes RASL11B, RASD1, GNG3, BAD, and BIK, comprises thymoquinone,
wherein said ferroptosis stimulation means for stimulating ferroptosis induced apoptosis in cancer cells comprises piperlongumine,
wherein said lysosomotropic means for increasing the permeability of lysosomes and enhancing apoptosis comprise chloroquine, hydroxychloroquine, loratadine, astemizole, ebastine, diphenhydramine and/or chlorpheniramine, and
wherein said means for enhancing pro-apoptotic TRAIL death receptor pathway comprises magnol and/or apigenin.

8. The method of claim 1 wherein the patient has fasted for at least 12 hours prior to administering said multiple apoptosis pathway drug means, further comprising briefly administering to the patient means for reducing phagocytic digestion of apoptosing cancer cells.

9. The screening method of claim 1 further comprising briefly administering to the patient before said step of obtaining a sample of the patient's body fluid, an effective amount of means for inhibiting degradation of ctDNA in the patient's bloodstream to increase the concentration of ctDNA, if present, for detection in the screening patient's blood and other body fluids.

10. The method of claim 9, further comprising the step of briefly administering to the patient means for reducing phagocytic digestion of apoptosing cancer cells to further briefly increase the concentration of ctDNA in the patient's bloodstream prior to obtaining said sample of said patient's bodily fluid.

11. A method for detecting early-stage human cancer in an asymptotic human cancer screening patient not previously diagnosed with cancer and who has free circulating bloodstream cfDNA including a natural amount and concentration of circulating tumor DNA (ctDNA) shed by some cancer cells of a malignant tumor if present within the patient, the cancer cells of which have impaired or inactivated apoptosis functionality which impairs their natural programmed cell death, comprising the steps of:
obtaining a first sample of a body fluid from the patient;
concurrently with obtaining said first sample, administering short-term safe, effective dosages to the cancer screening patient of multiple apoptosis pathway drug means for selectively reactivating or inducing apoptosis across multiple apoptosis pathways in human cancer cells to release ctDNA into the patient's bloodstream to increase the amount and concentration of ctDNA in the patient's bloodstream and other bodily fluids above the natural amount and concentration of ctDNA, without substantial harm to normal nonmalignant patient cells, wherein said multiple apoptosis pathway drug means comprises at least 3 apoptotic pathway means for reactivating or restoring apoptosis pathways which are downregulated or otherwise functionally deactivated or silenced in malignant human cancer cells, selected from:
p53 means for activating p53-induced apoptosis in cancer cells,
PDK inhibition means for inhibiting pyruvate dehydrogenase kinases to reduce aerobic glycolysis in cancer cells,
mTOR means for inhibiting the pI3K/Akt/mTOR signaling pathway,
Bax-Bcl2 ratio means for increasing the Bax:Blc-2 cancer cell ratio,
caspase expression means for increasing cancer cell caspase expression of caspase-3, caspase-7 or caspase-9,
DNA demethylation means for reducing cancer cell DNA hypermethylation,
tumor suppressor upregulation means for upregulating at least 2 of tumor suppressor genes DLC1, PPARG, ST7, FOXO6, TET2, CYP1B1, SALL4, and DDIT3, together with upregulation of at least two of pro-apoptotic genes RASL11B, RASD1, GNG3, BAD, and BIK,
histone demethylase inhibition means for inhibiting histone demethylase to restore functional cancer cell histone methylation for cancer cell apoptosis initiation,
ferroptosis stimulation means for stimulating ferroptosis induced apoptosis in cancer cells,
lysosomotropic means for increasing the permeability of lysosomes and enhancing apoptosis, and
means for enhancing pro-apoptotic TRAIL death receptor pathway;
discontinuing the administration of said multiple apoptosis pathway drug means for selectively inducing apoptosis in human cancer cells within 3 days of the initial administration;
obtaining a second sample of a body fluid from the patient during said increase in the amount and concentration of ctDNA in the patient's bloodstream and other bodily fluids within 7 days of the initial administration of the apoptosis inducing drug means; and
analyzing the first sample fluid from the patient and the second sample fluid from the patient for the presence of circulating tumor DNA and/or cancer-characteristic circulating biomarkers.
